(19) United States
(12) Patent Application Publication
(10) Pub. No.: US 2015/0167054 A1
(43) Pub. Date: Jun. 18, 2015

(54) METHOD OF IDENTIFICATION USING NUCLEIC ACID TAGS

(71) Applicant: GENEWORKS TECHNOLOGIES PTY LTD, THEBARTON, SOUTH AUSTRALIA (AU)

(72) Inventors: Robert King, Unley (AU); Peter Guilhaus, Lynton (AU)

(73) Assignee: Geneworks Technologies Pty Ltd a corporation

(21) Appl. No.: 14/412,975

(22) PCT Filed: Jul. 5, 2013
(86) PCT No.: PCT/AU2013/000730

§ 371 Date: Jan. 5, 2015

(30) Foreign Application Priority Data
Jul. 6, 2012 (AU) 2012902892

(51) Int. Cl. C12Q 1/68

(52) U.S. Cl. CPC C12Q 1/6806

(57) ABSTRACT

The present disclosure relates to methods of identification or marking using nucleic acid tags, isolated nucleic acids, marking compositions and the use of nucleic acid tags for identification or marking. Certain embodiments of the present disclosure provide a method of identifying an object marked with a nucleic acid tag, the nucleic acid tag comprising a plurality of selected different nucleic acids. The method comprises amplifying nucleic acids in the nucleic acid tag, wherein the amplification of the plurality of selected different nucleic acids in the nucleic acid tag produces a plurality of amplification products with a different size, detecting the plurality of selected different nucleic acids in the nucleic acid tag by the size of the plurality of amplification products, and identifying the object marked with the nucleic acid tag by the presence of the plurality of selected different nucleic acids in the nucleic tag.

### PRIORITY CLAIM

This application claims priority to Australian provisional patent application 2012902892 filed on 6 Jul. 2012, the content of which is hereby incorporated by reference.

### FIELD

The present disclosure relates to methods of identification using nucleic acid tags, nucleic acids for use in tags and isolated nucleic acids.

### BACKGROUND

There is a continuing need in the fields of commerce and security to provide marking and/or tracing systems. For example, many products are marked with a tag that allows the identity or source of the product to be determined. In other circumstances, products are marked with a tag as a means to allowing tracking of the product. In the security field, products are available that may be used to tag individuals who have may been involved in illegal activities such as theft and/or break-in. Typically, such products mark the individual(s) involved with a solution that adheres to the skin or clothing of the individual. The solution can then be detected at a later point in time to assist in establishing whether or not the individuals may have been involved in the illegal activities.

Aside from the uses described above, there are additional applications of marking and tracing systems. For example, such systems can be used to identify genuine products and distinguish them from counterfeit products, or to identify cases of parallel trading. Such marking and tracing systems may also be used to trace the path and/or timing of an object as it moves from one location to another.

There are also circumstances where it may be necessary to identify the source of a product, such as may occur in situations where a substance contaminates another product or environment.

Substances used to mark a product can be visible, such as a dye or coloured agent. They may also not be visible to the unaided eye, and only visible or detectable under certain conditions or by certain assays. Typically tags that are not visible are less subject to tampering. Examples of substances used to mark a product include inks, paints, photochromic compounds, luminescent agents, fluorescent agents and oligonucleotides.

While many substances used to tag products may not be visible to the unaided eye, the possible informational content of such substances is low which reduces their capacity to be used to distinguish a large number of products. Examples of such substances include fluorescent agents. On the other hand, printed barcodes represent a situation when informational content is very high and can be varied, but the barcodes are generally visible and printing of the bar code requires specialised equipment.

Oligonucleotides are not visible to the unaided eye and can provide effective tags, as they are easy to synthesize and their information content is also very high, meaning that a very large number of different specific oligonucleotides can potentially be produced and which can be tailored for specific circumstances. However, methods for determining the identity of oligonucleotides can be complicated, time consuming and/or expensive, particularly when the sequence of the oligonucleotide need to be determined.

Accordingly, there is a need for improved methods and compositions for marking and detecting objects, in order to address one or more problems in the art and/or to provide one or more advantages in the art.

### SUMMARY

The present disclosure relates to methods of identification and marking using nucleic acid tags, marking compositions, isolated nucleic acids and nucleic acid tags, and to the use of nucleic acid tags for identification and marking.

Certain embodiments of the present disclosure provide a method of identifying an object marked with a nucleic acid tag, the nucleic acid tag comprising a plurality of selected different nucleic acids, the method comprising:
amplifying nucleic acids in the nucleic acid tag, wherein the amplification of the plurality of selected different nucleic acids in the nucleic acid tag produces a plurality of amplification products with a different size;
detecting the plurality of selected different nucleic acids in the nucleic acid tag by the size of the plurality of amplification products; and
identifying the object marked with the nucleic acid tag by the presence of the plurality of selected different nucleic acids in the nucleic tag.

Certain embodiments of the present disclosure provide a method of identifying an object marked with a nucleic acid tag comprising a plurality of selected different nucleic acids, the method comprising:
sampling the object comprising the nucleic acid tag, wherein the selected different nucleic acids comprises a first primer region, a second primer region and an intervening region between the first primer region and the second primer region, the size of the intervening region being different between the selected different nucleic acids;
amplifying nucleic acids in the sample, the amplification comprising a primer to the first primer region and a primer to the second primer region;
determining the presence of the plurality of selected different nucleic acids by the size of the amplification products; and
identifying the object marked with the nucleic acid tag by the presence of the plurality of selected different nucleic acids in the nucleic acid tag.

Certain embodiments of the present disclosure provide a method of determining whether an object has been marked with a nucleic acid tag, the nucleic acid tag comprising a plurality of selected different nucleic acids, the method comprising:
obtaining a sample from the object;
amplifying nucleic acids in the sample, wherein amplification of the plurality of selected different nucleic acids in the nucleic acid tag produces a plurality of amplification products with a different size;
detecting the plurality of selected different nucleic acids in the nucleic acid tag by the size of the plurality of amplification products; and
determining whether the object has been marked with the nucleic acid tag by the presence of the plurality of selected different nucleic acids in the nucleic tag.

Certain embodiments of the present disclosure provide a method of marking an object with a nucleic acid tag, the method comprising:
selecting a plurality of different nucleic acids to mark the object, the different nucleic acids having a first primer region, a second primer region and an intervening region between the first primer region and the second primer region, wherein the size of the intervening region is different between the different nucleic acids; and adding the plurality of selected different nucleic acids to the object, thereby marking the object with a nucleic acid tag.

Certain embodiments of the present disclosure provide a plurality of isolated different nucleic acids, the plurality of different nucleic acids comprising:
a first primer region and a second primer region, wherein the nucleotide sequence of the first primer region and/or the nucleotide sequence of the second primer region comprise a sequence identity of 80% or less with a naturally occurring genomic nucleotide sequence; and
an intervening region between the first primer region and the second primer region; wherein the size of the intervening region is different between the different nucleic acids.

Certain embodiments of the present disclosure provide a marking composition, the composition comprising a plurality of isolated different nucleic acids, the plurality of isolated different nucleic acids comprising:
a first primer region and a second primer region, wherein the nucleotide sequence of the first primer region and/or the nucleotide sequence of the second primer region comprise a sequence identity of 80% or less with a naturally occurring genomic nucleotide sequence; and
an intervening region between the first primer region and the second primer region; wherein the size of the intervening region is different between the isolated different nucleic acids.

Certain embodiments of the present disclosure provide an isolated nucleic acid comprising:
a first primer region and/or a second primer region, the first primer region and/or the second primer region comprising a sequence identity of 80% or less with a naturally occurring genomic sequence; and
an intervening region between the first primer region and the second primer region, the intervening region having a size of at least 15 base pairs.

Certain embodiments of the present disclosure provide an isolated nucleic acid comprising:
(i) SEQ ID NO.1 and/or the complement thereof; and/or
(ii) a nucleotide sequence with greater than 80% sequence identity to SEQ ID NO.1 and/or the complement thereof.

Certain embodiments of the present disclosure provide an isolated nucleic acid comprising:
(i) SEQ ID NO.2 and/or the complement thereof; and/or
(ii) a nucleotide sequence with greater than 80% sequence identity to SEQ ID NO.2 and/or the complement thereof.

Certain embodiments of the present disclosure provide an isolated nucleic acid comprising:
(i) SEQ ID NO.3 and/or the complement thereof; and/or
(ii) a nucleotide sequence with greater than 80% sequence identity to SEQ ID NO.3 and/or the complement thereof.

Certain embodiments of the present disclosure provide an isolated nucleic acid comprising one or more of a nucleotide sequence selected from one of SEQ ID NOs. 30 to 60, or the complement of any one or more of the aforementioned nucleotide sequences.

Other embodiments are disclosed herein.

### BRIEF DESCRIPTION OF THE FIGURES

Certain embodiments are illustrated by the following figures. It is to be understood that the following description is for the purpose of describing particular embodiments only and/or for describing comparative examples, and is not intended to be limiting with respect to the description.

FIG. 1 shows the amplification products from individual plasmid templates. 200 fg of individual plasmid DNA templates were amplified using 6-FAM-labelled Primer 1 and Primer 2 in a 35 cycle PCR reactions using the AmpFISTR ProfilerPlus protocol, reaction mix and polymerase (Applied Biosystems). 4 ul of each reaction was loaded on a 2.5% agarose/TAE gel containing ethidium bromide. DMW-100L is a DNA marker containing 100-1000 bp DNA in 100 bp increments. Templates 100, 105, 110, 190, 290, 295 and 300 (2 different preparations) generated the expected 100, 105, 110, 190, 290, 295 and 300 bp products respectively.

FIG. 2 shows the effect on the PCR result of varying the amount of each template in a mixture, and the total amount of template present. Different amounts of 3 different ratio mixes of plasmid templates 100, 105, 110, 190, 290, 295 and 300 (see table at top of figure) were amplified using 6-FAM-labelled Primer 1, and Primer 2 in a 25 ul 35 cycle PCR reaction using AmpFISTR ProfilerPlus protocol, reaction mix and polymerase (Applied Biosystems). The products were analysed by 2 different methods: 6% non-denaturing polyacrylamide/TBE gel (left), same gel stained with ethidium bromide to determine PCR product sizes by visualisation of DNA size standards (centre), and 6% denaturing polyacrylamide/urea/TBE gel for improved size selection of 100-110 bp products (right).

FIG. 3 shows an estimation of the relative amounts of different sized templates required to produce a balanced PCR reaction yielding the same amount of each size product. For example, about 7 times more 300 bp template than 100 bp template is required to give the same amount of product. This analysis is based on the results shown in FIG. 2.

FIG. 4 shows the specificity of the PCR amplification from plasmid template in the presence of contaminating human, plant (lawn grass) and soil genomic DNA. Zero (NTC), 20 fg and 400 fg of both 100 and 300 plasmid template DNA was amplified using 6-FAM-labelled Primer 1, and Primer 2 in a 50 ul 35 cycle PCR reaction using AmpFISTR ProfilerPlus protocol, reaction mix and polymerase (Applied Biosystems). 4 ul of each reaction was loaded onto a 2.5% agarose/TAE gel stained with ethidium bromide.

FIG. 5 shows the effect of PCR amplification cycle number on the sensitivity of detection of plasmid template. Zero (NTC), 20 fg, 100 fg, 400 fg, 2000 fg of both 100 and 300 plasmid DNA was amplified using 6-FAM-labelled Primer 1 and Primer 2 in 28 or 35 cycle PCR reactions using the AmpFISTR ProfilerPlus protocol, reaction mix and polymerase (Applied Biosystems). 20 ul each sample was added to 50 ul 35 cycle reactions with AmpFISTR ProfilerPlus PCR mix. 4 ul of each PCR reaction was loaded onto 2.5% agarose/TAE gel stained with ethidium bromide.

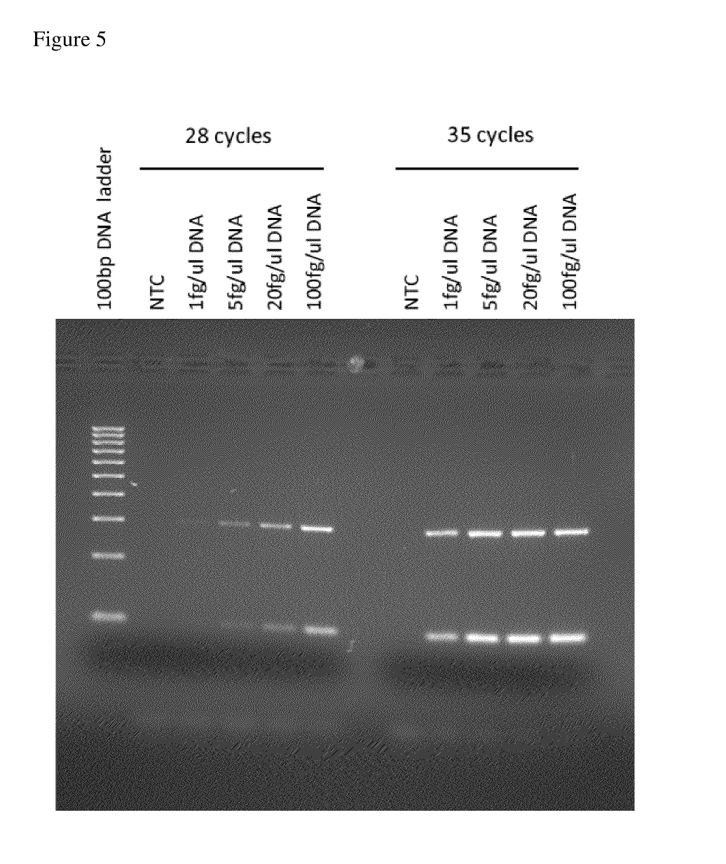

FIG. 6 shows the analysis of PCR amplification products by Capillary Electrophoresis on Applied Biosystems 3730 instrumentation (service provided by 1stBASE Pte Ltd, Singapore) from a mixture of plasmid templates. 200 fg each of templates 100, 105, 110, 190, 290, 295 and 300 were amplified using 6-FAM-labelled Primer 1, and Primer 2 in a 50 ul 35 cycle PCR reaction using AmpFISTR ProfilerPlus protocol, reaction mix and polymerase (Applied Biosystems).

FIG. 7 shows the reported fragment sizes from the analysis by by Capillary Electrophoresis on Applied Biosystems 3730xl instrumentation (service provided by 1stBASE Pte Ltd, Singapore) of individual and different mixtures of plasmid templates as indicated by analysis file name: 1=100, 2=105, 3=110, 4=190, 5=290, 6=295, 7 and 8=300 (different template preparations), A=95+100, B=95+105, C=100+105+190+290+295+300, D=95+105+190+290+300, E=190+300, F=95+105+290+300. 200 fg each template was amplified using 6-FAM-labelled Primer 1, and Primer 2 in a 50 ul 35 cycle PCR reaction using AmpFISTR ProfilerPlus protocol, reaction mix and polymerase (Applied Biosystems).

FIG. 8 shows the results of PCR amplification from swab samples taken at different times following spraying various items with plasmid template DNA. A mixture of 200 ng/ul each of plasmid templates 100 and 300, 1 mM Tris pH 8.0, 0.1 mM EDTA, pH 8.0 was sprayed onto the indicated items at Day 0 from approximately 1 metre distance. Cotton buds soaked in 200 ul of 10 mM Tris, pH8.0, 1 mM EDTA were used at various times after spraying to swab DNA from the sprayed surfaces. 20 ul of buffer surrounding the swab was then was amplified using 6-FAM-labelled Primer 1, and Primer 2 in a 50 ul 35 cycle PCR reaction using AmpFISTR ProfilerPlus protocol, reaction mix and polymerase (Applied Biosystems). 4 ul of each PCR reaction was loaded on a 2.5% agarose/TAE gel stained with ethidium bromide. A negative control (buffer from an unused swab) gave no products. A positive control containing 100 fg of each template was included to check reaction performance. Products of the expected size (100 bp and 300 bp) are detected by this method in all samples over the course of this study, though the amount generally declines with the time from exposure to spray on Day 0. Swab samples taken from human skin showed biased detection of the 100 bp template. There is some variability in the result between different sampling from the same surface, which is consistent with the expected non-uniform coverage by the sprayed DNA.

### DETAILED DESCRIPTION

The present disclosure relates to methods of identification or marking using nucleic acid tags, isolated nucleic acids, marking compositions and the use of nucleic acid tags for identification and marking.

Certain embodiments of the present disclosure provide a method of identifying an object marked with a nucleic acid tag.

Certain disclosed embodiments have one or more combinations of advantages. For example, some of the advantages of the embodiments disclosed herein include one or more of the following: an improved method of using nucleic acids for tagging; an improved method of using nucleic acids for security and identification purposes; an improved method of production of “barcoded” nucleic acids; an improved method of marking property; a method of non-visibly marking products and tradeable commodities; a flexible method of producing combinations of specific nucleic acids for use in tags; a method of tagging products that allows detection of very low amounts of a tag; a tag that is amenable for use in spray security devices; a method of tagging that is amenable for detection in many standard nucleic acid detection systems, including forensic detection; the ability to produce a very large number of different nucleic acid tags, including the ability to generate different classes and subclasses of nucleic acid tags; the ability to produce large amounts of the nucleic acid tags by utilising plasmid propagation; the ability to generate a library of different cloned nucleic acid tags, which allows different combinations of nucleic acids to be selected for a nucleic acid tag and/or which also assists in validation of the identity of the selected nucleic acids to be used in the nucleic acid tag; new isolated nucleic acids which are useful for tagging; to address one or more problems in the art; to provide one or more advantages; or to provide a useful commercial choice. Other advantages of certain embodiments are disclosed herein.

Certain embodiments of the present disclosure provide a method of identifying an object marked with a nucleic acid tag, the nucleic acid tag comprising a plurality of selected different nucleic acids, the method comprising:
amplifying nucleic acids in the nucleic acid tag, wherein the amplification of the plurality of selected different nucleic acids in the nucleic acid tag produces a plurality of amplification products with a different size;
detecting the plurality of selected different nucleic acids in the nucleic acid tag by the size of the plurality of amplification products; and
identifying the object marked with the nucleic acid tag by the presence of the plurality of selected different nucleic acids in the nucleic tag.

In certain embodiments, the object is a human. For example, a human may be marked with a nucleic tag when the human is marked with a tag when security procedures at a site are activated. Typically, an individual may be sprayed with a nucleic acid tag to mark the individual, and thereby mark the individual to assist in the identification at a later time. Certain embodiments of the present disclosure provide a method of identifying an individual marked with a nucleic acid tag. Certain embodiments of the present disclosure provide a nucleic acid tag for marking a human.

In certain embodiments, the object is an animal, plant or other organism, and/or a part thereof. For example, animals such as livestock and poultry can be marked with a nucleic acid tag that identifies the source of the animals, identifies their destination and/or is used to assist in tracking of the object. In certain embodiments, products derived from animals, plants or other organisms may be marked. Certain embodiments of the present disclosure provide a method of identifying an animal, plant or organism (or a part of any of these) with a nucleic acid tag. Certain embodiments of the present disclosure provide a nucleic acid tag for marking an animal, plant or other organism, and/or a part thereof.

In certain embodiments, the object comprises all or part of an article or a commodity. For example, the article may be a manufactured article marked with a nucleic acid tag that identifies the source of the article, identifies the destination of the article and/or is used to assist in tracking of the object. Examples of manufactured articles include cartons, packaging, currency, security documents, wrapping, paper, foils, inks, glues, and solvents.

Certain embodiments of the present disclosure provide a nucleic acid tag for marking an article. Nucleic acids for use in tags and nucleic acid tags are as described herein.

Certain embodiments of the present disclosure provide marking compositions, as described herein. Certain embodiments of the present disclosure provide a marking composition comprising one or more nucleic acids as described herein.

In certain embodiments, a nucleic acid tag or a marking composition as described herein comprises one or more non-nucleic acid agents. In certain embodiments, the nucleic acid tag or marking compositions as described herein comprises a stabilizer, a colourant, a fluorescent agent, a buffering agent and/or a metal ion chelator.

In certain embodiments, the nucleic acid tag or marking composition as described herein comprises one or more fluorescent agents. In certain embodiments, the nucleic acid tag or marking composition as described herein comprises a plurality of fluorescent agents. In certain embodiments, the fluorescent agent is not substantially visible under visible light. In certain embodiments, the fluorescent agent is visible under UV light. In certain embodiments, a fluorescent agent may be used to assist in identification of the object.

In certain embodiments, the methods of the present disclosure comprise sampling the object comprising the nucleic acid tag. Methods for sampling are known and include one or more of removal of part of the object, swabbing the object, and/or applying a solvent (such as water) to all or part of the object. In certain embodiments, sampling the object comprises swabbing the object. In certain embodiments, the methods of the present disclosure comprise obtaining a sample of nucleic acids in the nucleic acid tag from the object.

In certain embodiments, the methods of the present disclosure comprise identification of the nucleic tag in situ with the object. In certain embodiments, the method does not comprise sampling of the object.

The term “nucleic acid” refers to an oligonucleotide or a polynucleotide and includes for example DNA, RNA, DNA/RNA, a variant or DNA and/or RNA (for example a variant of the sugar-phosphate backbone and/or a variant of one or more bases, such as methylation), and may be single stranded, double stranded, non-methylated, methylated or other forms thereof. In certain embodiments, the nucleic acid is a non-naturally occurring nucleic acid, a naturally occurring nucleic acid, a nucleic acid of genomic origin, a mitochondrial nucleic acid, a nucleic acid of cDNA origin (derived from a mRNA), a nucleic acid derived from a virus, a nucleic acid of synthetic origin, a single stranded DNA, a double stranded DNA, an analogue of DNA and/or RNA, and/or a derivative, fragment and/or combination of any of the aforementioned. Examples of derivatives also include nucleic acids that have a blocking group at the 5′ and/or 3′ ends for example to improve stability, and/or nucleic acids fused to other molecules. Other types of nucleic acids are contemplated. Methods for producing nucleic acids are known and include for example nucleic acids produced by recombinant DNA technology or nucleic acids produced by chemical synthesis.

The term “nucleic acid” also refers to a specified nucleic acid or a nucleic acid comprising a nucleotide sequence which is the complement of the nucleic acid, a nucleic acid comprising a nucleotide sequence with greater than 70%, 75%, 80%, 85%, 90% or 95% sequence identity to the specified nucleic acid, or a nucleic acid comprising a nucleotide sequence with greater than 70%, 75%, 80%, 85%, 90% or 95% sequence identity to the complement of the specified nucleic acid. Other levels of sequence identity are contemplated.

The term “nucleic acid tag” refers to one or more nucleic acids which are used to mark an object.

In certain embodiments, the nucleic acid tag comprises a plurality of different nucleic acids. In certain embodiments, the nucleic acid tag comprises one or more selected nucleic acids. In certain embodiments, the nucleic acid tag comprises a plurality of selected nucleic acids. In certain embodiments, the nucleic acid tag comprises a plurality of selected different nucleic acids. In certain embodiments, the nucleic acid tag comprises one or more nucleic acids as described herein.

The term “selected different nucleic acid” refers to a nucleic acid which has been selected so as to be distinguishable from another nucleic acid. In certain embodiments, the selected different nucleic acids are distinguishable by size. In certain embodiments, the selected different nucleic acids are distinguishable by their amplification products. In certain embodiments, the selected different nucleic acids are distinguishable after amplification, for example as the amplification products are distinguishable by size. Other methods of directly and/or indirectly distinguishing the nucleic acids are contemplated.

In certain embodiments, one or more of the selected different nucleic acids is a DNA molecule. In certain embodiments, one or more of the selected different nucleic acids is a double stranded DNA molecule. Other types nucleic acid molecules are contemplated, and mixtures of nucleic acid molecules such as DNA and RNA are also contemplated. In certain embodiments, the plurality of selected different nucleic acids is a plurality of DNA molecules. In certain embodiments, the plurality of selected different nucleic acids is a plurality of double stranded DNA molecules.

In certain embodiments, the plurality of selected different nucleic acids comprises at least two different nucleic acids. In certain embodiments, the plurality of selected different nucleic acids comprises three, four, five, six, or more different nucleic acids. In certain embodiments, the plurality of selected different nucleic acids comprises at least three, at least four, at least five, or at least six different nucleic acids. Other numbers of different nucleic acids are contemplated.

In certain embodiments, one or more of the selected different nucleic acids have a size of 500 base pairs or less, 400 bp or less, 300 bp or less, 200 bp or less, 100 bp or less, or combinations thereof. Other sizes are contemplated. For example, one or more of the nucleic acids may have a size of greater than 500 base pairs.

In certain embodiments, one or more of the selected different nucleic acids have a size of 500 base pairs or less. In certain embodiments, the selected different nucleic acids have a size of 500 base pairs or less. In certain embodiments, the plurality of selected different nucleic acids have a size of 500 base pairs or less. In certain embodiments, one or more of the selected different nucleic acids have a size of 400 base pairs or less. In certain embodiments, the selected different nucleic acids have a size of 400 base pairs or less. In certain embodiments, the plurality of selected different nucleic acids have a size of 400 base pairs or less. In certain embodiments, one or more of the selected different nucleic acids have a size of 300 base pairs or less. In certain embodiments, the selected different nucleic acids have a size of 300 base pairs or less. In certain embodiments, the plurality of selected different nucleic acids have a size of 300 base pairs or less. In certain embodiments, one or more of the selected different nucleic acids have a size of 200 base pairs or less. In certain embodiments, the selected different nucleic acids have a size of 200 base pairs or less. In certain embodiments, the plurality of selected different nucleic acids have a size of 200 base pairs or less. In certain embodiments, one or more of the selected different nucleic acids have a size of 100 base pairs or less. In certain embodiments, the selected different nucleic acids have a size of 100 base pairs or less. In certain embodiments, the plurality of selected different nucleic acids have a size of 100 base pairs or less. Other sizes are contemplated.

In certain embodiments, one or more of the selected different nucleic acids have a size in the range from 100 to 500, 100 to 400, 100 to 300, 100 to 200, 200 to 500, 200 to 400, 200 to 300, 300 to 500, 300 to 400, or 400 to 500 base pairs.

In certain embodiments, the selected different nucleic acids have a size in the range from 100 to 500, 100 to 400, 100 to 300, 100 to 200, 200 to 500, 200 to 400, 200 to 300, 300 to 500, 300 to 400, or 400 to 500 base pairs. In certain embodiments, the plurality of selected different nucleic acids have a size in the range from 100 to 500, 100 to 400, 100 to 300, 100 to 200, 200 to 500, 200 to 400, 200 to 300, 300 to 500, 300 to 400, or 400 to 500 base pairs.

In certain embodiments, one or more of the selected different nucleic acids have a size in the range from 100 to 300 base pairs. In certain embodiments, the selected different nucleic acids have a size in the range from 100 to 300 base pairs. In certain embodiments, the plurality of selected different nucleic acids have a size in the range from 100 to 300 base pairs.

In certain embodiments, one or more of the selected different nucleic acids differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, or at least 1 base pair.

In certain embodiments, the selected different nucleic acids differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, or at least 1 base pair. In certain embodiments, the plurality of selected different nucleic acids differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, or at least 1 base pair.

It will also be appreciated that some methods of detection of nucleic acids, for example mass spectrometry, are able to distinguish sizes of nucleic acids by less than a single base pair. For example, some methods of detection are able to readily distinguish between the size of a nucleic acid that is non-methylated and one which is methylated. Accordingly, the present disclosure contemplates the use of such differences in size. In certain embodiments, one or more of the selected different nucleic acids differ in size by less than 1 base pair. In certain embodiments, the selected different nucleic acids differ in size by less than 1 base pair. In certain embodiments, the plurality of selected different nucleic acids differ in size by less than 1 base pair.

In certain embodiments, one or more of the selected different nucleic acids differ in size by at least 5 base pairs. In certain embodiments, the selected different nucleic acids differ in size by at least 5 base pairs. In certain embodiments, the plurality of selected different nucleic acids differ in size by at least 5 base pairs. In certain embodiments, one or more of the selected different nucleic acids differ in size by at least 1 base pair. In certain embodiments, the selected different nucleic acids differ in size by at least 1 base pair. In certain embodiments, the plurality of selected different nucleic acids differ in size by at least 1 base pair.

In certain embodiments, one or more of the selected different nucleic acids differ in size by less than 1 base pair. In certain embodiments, the selected different nucleic acids differ in size by less than 1 base pair. In certain embodiments, the plurality of selected different nucleic acids differ in size by less than 1 base pair.

In certain embodiments, one or more of the selected different nucleic acids comprises a first primer region, being a region for hybridization of a first primer to initiate nucleic acid synthesis using a polymerase. In certain embodiments, the selected different nucleic acids comprise a first primer region. In certain embodiments, the plurality of selected different nucleic acids comprises a first primer region.

In certain embodiments, the first primer region is identical between at least two of the selected different nucleic acids. In certain embodiments, the first primer region is identical between at least two of the plurality of selected different nucleic acids. In certain embodiments, the first primer region is identical between the plurality of selected different nucleic acids.

In certain embodiments, one or more of the selected different nucleic acids comprises a second primer region, being a region for hybridization of a second primer to initiate nucleic acid synthesis using a polymerase. In certain embodiments, the selected different nucleic acids comprise a second primer region. In certain embodiments, the plurality of selected different nucleic acids comprises a second primer region.

In certain embodiments, the second primer region is identical between at least two of the plurality of selected different nucleic acids. In certain embodiments, the second primer region is identical between the selected different nucleic acids. In certain embodiments, the second primer region is identical between the plurality of selected different nucleic acids.

In certain embodiments, the first primer region and the second primer region have an identical or substantially identical nucleotide sequence.

In certain embodiments, the first primer region and the second primer region have a different nucleotide sequence.

In certain embodiments, one or more of the plurality of selected different nucleic acids comprise a first primer region and/or a second primer region. In certain embodiments, one or more of the plurality of selected different nucleic acids comprise a first primer region and a second primer region.

In certain embodiments, the selected different nucleic acids comprise a first primer region and/or a second primer region. In certain embodiments, the selected different nucleic acids comprise a first primer region and a second primer region. In certain embodiments, the plurality of selected different nucleic acids comprise a first primer region and/or a second primer region. In certain embodiments, the plurality of selected different nucleic acids comprise a first primer region and a second primer region.

In certain embodiments, one or more of the plurality of selected different nucleic acids comprise a first primer region and/or a second primer region and the nucleotide sequence of the first primer region and/or the nucleotide sequence of the second primer region comprise a sequence identity of 90% or less, 85% or less, 80% or less, 75% or less, 70% or less, or 65% or less with a naturally occurring nucleotide sequence. In certain embodiments, the selected different nucleic acids comprise a first primer region and/or a second primer region and the nucleotide sequence of the first primer region and/or the nucleotide sequence of the second primer region comprise a sequence identity of 90% or less, 85% or less, 80% or less, 75% or less, 70% or less, or 65% or less with a naturally occurring nucleotide sequence. In certain embodiments, the plurality of selected different nucleic acids comprise a first primer region and/or a second primer region and the nucleotide sequence of the first primer region and/or the nucleotide sequence of the second primer region comprise a sequence identity of 90% or less, 85% or less, 80% or less, 75% or less, 70% or less, or 65% or less with a naturally occurring nucleotide sequence. In certain embodiments, the naturally occurring nucleotide sequence is a naturally occurring genomic sequence.

The term “genomic sequence” refers to the sequence of the genome of an organism, including exons, introns and any other DNA, such as non-coding DNA. For example, a “genomic sequence” comprises human genomic sequence, an animal genomic sequence, a genomic sequence from a microorganism such as a bacterium, or any one or more of the aforementioned. The genomic sequence may or may not include mitochondrial DNA.

In certain embodiments, the selected different nucleic acids comprise a first primer region and a second primer region, wherein the nucleotide sequence of the first primer region and/or the nucleotide sequence of the second primer region comprise a sequence identity of 80% or less with a naturally occurring nucleotide sequence. Other levels of sequence identity are contemplated and are as described herein. In certain embodiments, the plurality of selected different nucleic acids comprise a first primer region and a second primer region, wherein the nucleotide sequence of the first primer region and/or the nucleotide sequence of the second primer region comprise a sequence identity of 80% or less with a naturally occurring nucleotide sequence. Other levels of sequence identity are contemplated and are as described herein. In certain embodiments, the naturally occurring nucleotide sequence is a naturally occurring genomic sequence.

In certain embodiments, the selected different nucleic acids comprise a first primer region and/or a second primer region, wherein the nucleotide sequence of the first primer region and/or the nucleotide sequence of the second primer region comprise a sequence that is not identical with a naturally occurring genomic nucleotide sequence. In certain embodiments, the plurality of selected different nucleic acids comprise a first primer region and/or a second primer region, wherein the nucleotide sequence of the first primer region and/or the nucleotide sequence of the second primer region comprise a sequence that is not identical with a naturally occurring genomic nucleotide sequence.

In certain embodiments, the selected different nucleic acids comprise a first primer region and a second primer region, wherein the nucleotide sequence of the first primer region and/or the nucleotide sequence of the second primer region comprise a sequence identity of 80% or less with a naturally occurring genomic nucleotide sequence. In certain embodiments, the plurality of selected different nucleic acids comprise a first primer region and a second primer region, wherein the nucleotide sequence of the first primer region and/or the nucleotide sequence of the second primer region comprise a sequence identity of 80% or less with a naturally occurring genomic nucleotide sequence.

In certain embodiments, the nucleotide sequence of the first primer region comprises SEQ ID NO.1, the complement of SEQ ID NO.1 or a nucleotide sequence with greater than 80% sequence identity to either of the aforementioned nucleotide sequences. Other levels of sequence identity are contemplated and are as described herein. In certain embodiments, the nucleotide sequence of the first primer region is SEQ ID NO.1, the complement of SEQ ID NO.1 or a nucleotide sequence with greater than 80% sequence identity to either of the aforementioned nucleotide sequences.

The nucleotide sequence of SEQ ID NO.1 is as follows: 5′ CCGCTGACAAGTAAACGCGATTGAT 3′ (SEQ ID NO.1).

In certain embodiments, the nucleotide sequence of the first primer region comprises the following nucleotide sequence:

5′ NNGCTGACAAGTAAACGCGATTGNN 3′ (SEQ ID NO.30), wherein N is A, C, G, T or no nucleotide, or the complement of SEQ ID NO.30.

In certain embodiments, the nucleotide sequence of the first primer region comprises a nucleotide sequence selected from one of following nucleotide sequences, or the complement of one of the following nucleotide sequences:

(SEQ ID NO. 1)
5′ CCGCTGACAAGTAAACGCGATTGAT 3′;
wherein N is A, C, G, T
(SEQ ID NO. 30)
5′ NNGCTGACAAGTAAACGCGATTGNN 3′,
or
no nucleotide;
(SEQ ID NO. 31)
5′ CCGCTGACAAGTAAACGCGATTGAT 3′;
(SEQ ID NO. 32)
5′ CGCTGACAAGTAAACGCGATTGAT 3′;
(SEQ ID NO. 33)
5′ GCTGACAAGTAAACGCGATTGAT 3′;
(SEQ ID NO. 34)
5′ CCGCTGACAAGTAAACGCGATTGA 3′;
(SEQ ID NO. 35)
5′ CCGCTGACAAGTAAACGCGATTG 3′;
(SEQ ID NO. 36)
5′ CGCTGACAAGTAAACGCGATTGA 3′;
(SEQ ID NO. 37)
5′ CGCTGACAAGTAAACGCGATTG 3′;
(SEQ ID NO. 38)
5′ GCTGACAAGTAAACGCGATTGA 3′;
(SEQ ID NO. 39)
5′ GCTGACAAGTAAACGCGATTGA 3′;
(SEQ ID NO. 40)
5′ GCTGACAAGTAAACGCGATTG 3′;
(SEQ ID NO. 41)
5′ (G/C)(G/C)GCTGACAAGTAAACGCGATTG(A/T)(A/T) 3′;
(SEQ ID NO. 42)
5′ (G/C)GCTGACAAGTAAACGCGATTG(A/T)(A/T) 3′;
(SEQ ID NO. 43)
5′ GCTGACAAGTAAACGCGATTG(A/T)(A/T) 3′;
(SEQ ID NO. 44)
5′ (G/C)(G/C)GCTGACAAGTAAACGCGATTG(A/T) 3′;
and
(SEQ ID NO. 45)
5′ (G/C)(G/C)GCTGACAAGTAAACGCGATTG 3′.

In certain embodiments, the nucleotide sequence of the second primer region comprises SEQ ID NO.2, the complement of SEQ ID NO.2 or a nucleotide sequence with greater than 80% sequence identity to either of the aforementioned nucleotide sequences. Other levels of sequence identity are contemplated and are as described herein. In certain embodiments, the nucleotide sequence of the second primer region is SEQ ID NO.2, the complement of SEQ ID NO.2 or a nucleotide sequence with greater than 80% sequence identity to either of the aforementioned nucleotide sequences. The nucleotide sequence of SEQ ID NO.2 is as follows: 5′ TGCCTACTGAAAAGTCGGACCATAG 3′ (SEQ ID NO.2).

In certain embodiments, the nucleotide sequence of the second primer region comprises the following nucleotide sequence:

5′ NNCCTACTGAAAAGTCGGACCATNN 3′ (SEQ ID NO.46), wherein N is A, C, G, T or no nucleotide, or the complement of SEQ ID NO.46.

In certain embodiments, the nucleotide sequence of the second primer region comprises a nucleotide sequence selected from one of following nucleotide sequences, or the complement of one of the following sequences:

(SED ID NO. 2)
5′ TGCCTACTGAAAAGTCGGACCATAG 3′;
wherein N is A, C, G, T
(SEQ ID NO. 46)
5′ NNCCTACTGAAAAGTCGGACCATNN 3′,
or
no nucleotide,
or
the complement of SEQ ID NO. 46;
(SED ID NO. 47)
5′ GCCTACTGAAAAGTCGGACCATAG 3′;
(SED ID NO. 48)
5′ CCTACTGAAAAGTCGGACCATAG 3′;
(SED ID NO. 49)
5′ TGCCTACTGAAAAGTCGGACCATA 3′;
(SED ID NO. 50)
5′ TGCCTACTGAAAAGTCGGACCAT 3′;
(SED ID NO. 51)
5′ GCCTACTGAAAAGTCGGACCATA 3′;
(SED ID NO. 52)
5′ GCCTACTGAAAAGTCGGACCAT 3′;
(SED ID NO. 53)
5′ GCCTACTGAAAAGTCGGACCAT 3′;
(SED ID NO. 54)
5′ CCTACTGAAAAGTCGGACCATA 3′;
(SED ID NO. 55)
5′ CCTACTGAAAAGTCGGACCAT 3′;
(SEQ ID NO. 56)
5′ (T/A)(G/C)CCTACTGAAAAGTCGGACCAT(A/T)(G/C) 3′;
(SEQ ID NO. 57)
5′ (G/C)CCTACTGAAAAGTCGGACCAT(A/T)(G/C) 3′;
(SEQ ID NO. 58)
5′ CCTACTGAAAAGTCGGACCAT(A/T)(G/C) 3′;
(SEQ ID NO. 59)
5′ (T/A)(G/C)CCTACTGAAAAGTCGGACCAT(A/T) 3′;
and
(SEQ ID NO. 60)
5′ (T/A)(G/C)CCTACTGAAAAGTCGGACCAT 3′.

In certain embodiments, the nucleotide sequence of the first primer region comprises a nucleotide sequence selected from (i) SEQ ID NO.1, the complement of SEQ ID NO.1 or a nucleotide sequence with greater than 80% sequence identity to either of the aforementioned, and/or (ii) a nucleotide sequence selected from one of SEQ ID NOs. 30 to 45, and/or the complement of one of the aforementioned nucleotide sequences.

In certain embodiments, the nucleotide sequence of the second primer region comprises a nucleotide sequence selected from (i) SEQ ID NO.2, the complement of SEQ ID NO.2 or a nucleotide sequence with greater than 80% sequence identity to either of the aforementioned, and/or (ii) a nucleotide sequence selected from one of SEQ ID NOs. 46 to 60, and/or the complement of one of the aforementioned nucleotide sequences.

In certain embodiments, one or more of the plurality of selected different nucleic acids comprise an intervening region between the first primer region and the second primer region. As described herein, the first primer region and the second primer region may be identical, substantially identical or different. In certain embodiments, the selected different nucleic acids comprise an intervening region between the first primer region and the second primer region. In certain embodiments, the plurality of selected different nucleic acids comprises an intervening region between the first primer region and the second primer region.

In certain embodiments, the nucleotide sequence and/or size of the intervening region is different between one or more of the selected different nucleic acids. In certain embodiments, the nucleotide sequence and/or size of the intervening region is different between the selected different nucleic acids. In certain embodiments, the nucleotide sequence and/or size of the intervening region is different between the plurality of selected different nucleic acids.

In certain embodiments, the size of the intervening region is variable between one or more of the selected different nucleic acids. In certain embodiments, the size of the intervening region is variable between the selected different nucleic acids. In certain embodiments, the size of the intervening region is variable between the plurality of selected different nucleic acids.

In certain embodiments, the selected different nucleic acids comprise an intervening region between the first primer region and the second primer region; wherein the size of the intervening region is different between the selected different nucleic acids. In certain embodiments, the plurality of selected different nucleic acids comprise an intervening region between the first primer region and the second primer region; wherein the size of the intervening region is different between the selected different nucleic acids.

In certain embodiments, one or more of the plurality of selected different nucleic acids comprises a cleavage site for a restriction endonuclease between the first primer region and the second primer region. In certain embodiments, the selected different nucleic acids comprise a cleavage site for a restriction endonuclease between the first primer region and the second primer region. In certain embodiments, the plurality of selected different nucleic acids comprises one or more cleavage sites for a restriction endonuclease between the first primer region and the second primer region.

In certain embodiments, the cleavage site comprises a site for a restriction endonuclease with a least an 8 base specificity. In certain embodiments, the cleavage site comprises a site for a restriction endonuclease with at least a 6 base specificity. In certain embodiments, the cleavage site comprises a site for a restriction endonuclease with at least a 4 base specificity. In certain embodiments, the cleavage site comprises a site for a restriction endonuclease that is not elsewhere present in one or more of the selected nucleic acids. In certain embodiments, the cleavage site comprises an AflII restriction site. Other restriction sites are contemplated.

In certain embodiments, the intervening region comprises a nucleotide sequence with a low potential to form a secondary structure. Methods for assessing the potential of nucleic acids to form regions of secondary structure are known.

In certain embodiments, the intervening region comprises a size of at least 10 base pairs, at least 15 base pairs, at least 50 base pairs, at least 100 base pairs, at least 150 base pairs, at least 200 base pairs, at least 215 base pairs, at least 240 base pairs, at least 250 base pairs, at least 300 base pairs, at least 400 base pairs or at least 500 base pairs. Other sizes are contemplated.

In certain embodiments, the intervening region comprises a size of 10 base pairs or less, 15 base pairs or less, 50 base pairs or less, 100 base pairs or less, 150 base pairs or less, 200 base pairs or less, 215 base pairs or less, 240 base pairs or less, 250 base pairs or less, 300 base pairs or less, 400 base pairs or less, or 500 base pairs or less. Other sizes are contemplated.

In certain embodiments, the intervening region comprises a size of 10-500, 10-400, 10-300, 10-250, 10-240, 10-215, 10-200, 10-150, 10-100, 10-50, 15-500, 15-400, 15-300, 15-250, 15-240, 15-215, 15-200, 15-150, 15-100, 15-50, 50-500, 50-400, 50-300, 50-250, 50-240, 50-215, 50-200, 50-150, 50-100, 10-500, 100-400, 100-300, 100-250, 100-240, 100-215, 100-200, 100-150, 150-500, 150-400, 150-300, 150-250, 150-240, 150-215, 150-200, 200-500, 200-400, 200-300, 200-250, 200-240, 200-215, 215-500, 215-400, 215-300, 215-250, 215-240, 240-500, 240-400, 240-300, 240-250, 250-500, 250-400, 250-300, 300-500, 300-400, or 400-500 base pairs. Other sizes are contemplated.

In certain embodiments, the intervening region comprises a size of 15-240 base pairs, or a size of 15-215 base pairs.

In certain embodiments, the nucleotide sequence of the intervening region comprises SEQ ID NO.3, the complement of SEQ ID NO.3 or a nucleotide sequence with greater than 80% sequence identity to either of the aforementioned nucleotide sequences. In certain embodiments, the nucleotide sequence of the intervening region is SEQ ID NO.3, the complement of SEQ ID NO.3 or a nucleotide sequence with greater than 80% sequence identity to either of the aforementioned nucleotide sequences.

The nucleotide sequence of SEQ ID NO.3 is as follows: 5′-CACATTTTGGTGAGAAGTTGTCCAGACTTAAGGACAGTGATTCAAGACGTTCTAG TCGGTCCCAAACCTGACATGCATGGGCGCGCAACCTCTAAACCTTAACTCAGAA GTGAGTGCTTCCTGGAACCTCTAGTCTGGATCCCGGCAGCTACCTCGGACGGTTG AGAGGAGTACCCTAGTTCGCTGCCAGGCGTATATCCCGCAATCAACATCCCTGTA TAAATTAGTTTGGATATTTCAACATATTG-3′ (SEQ ID NO.3).

In certain embodiments, one or more of the plurality of selected different nucleic acids comprises one or more regions of shared sequence between primer regions. In certain embodiments, the selected different nucleic acids comprise one or more regions of shared sequence between primer regions. In certain embodiments, the plurality of selected different nucleic acids comprises one or more regions of shared sequence between primer regions. In certain embodiments, the plurality of selected different nucleic acids comprises one or more regions of shared sequence between primer regions.

In certain embodiments, one or more of the plurality of selected different nucleic acids comprises one or more regions of shared sequence between a first primer region and a second primer region. In certain embodiments, the selected different nucleic acids comprise one or more regions of shared sequence between a first primer region and a second primer region. In certain embodiments, the plurality of selected different nucleic acids comprises one or more regions of shared sequence between a first primer region and a second primer region.

In certain embodiments, the region of shared sequence comprises a nucleotide sequence with a low potential to form a secondary structure.

In certain embodiments, the region of shared sequence comprises a size of at least 1, 5, 10, 15, 20, 25, 30, 35, 40, 45 or 50 base pairs. In certain embodiments, the region of shared sequence comprises a size of 1, 5 or less, 10 or less, 15 or less, 20 or less, 25 or less, 30 or less, 35 or less, 40 or less, 45 or less, or 50 or less base pairs.

In certain embodiments, the region of shared sequence comprises a size of 1-50, 1-45, 1-40, 1-35, 1-30, 1-25, 1-20, 1-15, 1-10, 1-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-45, 30-35, 35-50, 35-45, 35-40, 40-50, or 45-50 base pairs.

In certain embodiments, the region of shared sequence comprises a length of 1 to 50 base pairs. In certain embodiments, the region of shared sequence comprises a size of 10-50 base pairs. In certain embodiments, the region of shared sequence comprises a size of 35 base pairs. Other sizes are contemplated.

In certain embodiments, the nucleotide sequence of the region of shared sequence comprises SEQ ID NO.4, the complement of SEQ ID NO.4 or a nucleotide sequence with greater than 80% sequence identity to either of the aforementioned nucleotide sequences. Other levels of sequence identity are contemplated and are as described herein. In certain embodiments, the nucleotide sequence of the region of shared sequence is SEQ ID NO.4, the complement of SEQ ID NO.4 or a nucleotide sequence with greater than 80% sequence identity to either of the aforementioned nucleotide sequences. The nucleotide sequence of SEQ ID NO.4 is as follows: 5′ CACATTTTGGTGAGAAGTTGTCCAGACTTAAGGAC 3′ (SEQ ID NO.4).

In certain embodiments, one or more of the regions of shared sequence comprises one or more cleavage sites for a restriction endonuclease. In certain embodiments, a cleavage site comprises a site for a restriction endonuclease with a least an 8 base specificity. In certain embodiments, a cleavage site comprises a site for a restriction endonuclease with at least a 6 base specificity. In certain embodiments, a cleavage site comprises a site for a restriction endonuclease with at least a 4 base specificity. In certain embodiments, a cleavage site comprises a site for a restriction endonuclease that is not elsewhere present in one or more of the selected nucleic acids. In certain embodiments, a cleavage site comprises an AflII restriction site. Other restriction sites are contemplated.

The term “amplifying”, or variants thereof, refers to the production of additional copies of a nucleic acid sequence. Methods for amplifying nucleic acids are known and are, for example, described in Sambrook et al. Molecular Cloning: A Laboratory Manual. 3rd ed. Cold Spring Harbor Laboratory Press, New York, N.Y. 2000 or Current Protocols in Molecular Biology. Ed. Ausubel et al. John Wiley & Sons, Inc. Cambridge, Mass., 2000. Methods include for example polymerase chain reaction and other methods of amplification, such as “loop-mediated isothermal amplification” which is as described for example in Notomi T. et al (2000) “Loop-mediated isothermal amplification of DNA” Nucleic Acids Research 28:E63 and rolling circle amplification on circular templates, for example as described in Fire, A. and Xu, S-Q. (1995) Proc. Natl. Acad. Sci 92:4641-4645. Other methods of amplification are contemplated.

In certain embodiments, amplification comprises one or more other steps, such as a step of producing a cDNA strand from an RNA, for example by using a reverse transcriptase.

In certain embodiments, the amplification of nucleic acids in the nucleic acid tag comprises amplification of one or more of the plurality of selected different nucleic acids. In certain embodiments, the amplification of nucleic acids in the nucleic acid tag comprises amplification of the selected different nucleic acids. In certain embodiments, the amplification of nucleic acids in the nucleic acid tag comprises amplification of the plurality of selected different nucleic acids.

In certain embodiments, the amplifying of the nucleic acids in the nucleic acid tag (and thereby the amplification of one or more of the plurality of selected different nucleic acids if present in the nucleic acid tag) comprises a polymerase chain reaction. In certain embodiments, the amplification comprises real-time quantitative polymerase chain reaction (qPCR). Other variants of the polymerase chain reaction are contemplated and are known.

In certain embodiments, the methods of the present disclosure comprise amplifying of the nucleic acids in the nucleic acid tag by a polymerase chain reaction. Methods for polymerase chain reaction are known and are as described herein. In certain embodiments, a polymerase chain reaction comprises use of a high fidelity polymerase and/or the use of a polymerase with increased processivity. Polymerases for use in polymerase chain reaction are known and commercially available.

In certain embodiments, the amplifying of the selected different nucleic acids in the nucleic acid tag produces a plurality of amplification products with a different size. In certain embodiments, the amplifying of the plurality of selected different nucleic acids in the nucleic acid tag produces a plurality of amplification products with a different size.

In certain embodiments, the amplifying comprises a first primer comprising a nucleotide sequence selected from (i) SEQ ID NO.1, the complement of SEQ ID NO.1, or a nucleotide sequence with greater than 80% sequence identity to either of the aforementioned nucleotide sequences, and/or (ii) a nucleotide sequence selected from one of SEQ ID NOs. 30 to 45 or the complement of one of the aforementioned nucleotide sequences, and/or a second primer comprising a nucleotide sequence selected from (i) SEQ ID NO.2, the complement of SEQ ID NO.2, or a nucleotide sequence with greater than 80% sequence identity to either of the aforementioned nucleotide sequences, and/or (ii) a nucleotide sequence selected from one of SEQ ID NOs. 46 to 60, or the complement of one of the aforementioned nucleotide sequences. It will be appreciated that other levels of sequence identity are contemplated for the aforementioned nucleotide sequences and are as described herein.

In certain embodiments, one or more of the plurality of amplification products have a size of 500 base pairs or less, 400 bp or less, 300 bp or less, 200 bp or less, or 100 bp or less.

In certain embodiments, one or more of the plurality of amplification products have a size of 500 base pairs or less. In certain embodiments, the plurality of amplification products have a size of 500 base pairs or less. In certain embodiments, one or more of the plurality of amplification products have a size of 400 base pairs or less. In certain embodiments, the plurality of amplification products have a size of 400 base pairs or less. In certain embodiments, one or more of the plurality of amplification products have a size of 300 base pairs or less. In certain embodiments, the plurality of amplification products have a size of 300 base pairs or less. In certain embodiments, the plurality of amplification products have a size of 200 base pairs or less. In certain embodiments, the plurality of amplification products have a size of 200 base pairs or less. In certain embodiments, the plurality of amplification products have a size of 100 base pairs or less. In certain embodiments, the plurality of amplification products have a size of 100 base pairs or less.

In certain embodiments, one or more of the plurality of amplification products have a size in the range from 100 to 500, 100 to 400, 100 to 300, 100 to 200, 200 to 500, 200 to 400, 200 to 300, 300 to 500, 300 to 400, or 400 to 500 base pairs. In certain embodiments, the plurality of amplification products have a size in the range from 100 to 500, 100 to 400, 100 to 300, 100 to 200, 200 to 500, 200 to 400, 200 to 300, 300 to 500, 300 to 400, or 400 to 500 base pairs. Other sizes are contemplated.

In certain embodiments, the plurality of amplification products have a size in the range from 100 to 300 base pairs.

In certain embodiments, one or more of the plurality of amplification products differ in size by at least 50, at least 45, at least 40, at least 35, at least 30, at least 25, at least 20, at least 15, at least 10, or at least 5 base pairs.

In certain embodiments, the plurality of amplification products differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, or at least 1 base pair.

In certain embodiments, the plurality of amplification products differ in size by at least 5 base pairs. In certain embodiments, the plurality of amplification products differ in size by at least 1 base pair. In certain embodiments, the plurality of amplification products differ in size by less than 1 base pair.

In certain embodiments, the amplification of nucleic acids in the nucleic acid tag (and thereby the amplification of one or more of the plurality of selected different nucleic acids if present in the nucleic acid tag) comprises at least one primer. In certain embodiments, the amplification of nucleic acids in the nucleic acid tag comprises a single primer. For example, amplification by a rolling circle method may utilise a single primer, or a single primer may be used in polymerase chain reaction to amplify a target nucleic acid, if the primer is able to hybridise to the same target sequence on each strand of the target nucleic acid.

The term “primer” refers to a nucleic acid, being an oligonucleotide or polynucleotide, that may be used to initiate nucleic acid synthesis using a polymerase under suitable conditions. Examples of nucleic acids are as described herein. For example, a primer may be a DNA, a RNA, a DNA/RNA, or a variant thereof. In certain embodiments, the primer may be labelled, for example by being fluorescently labelled. Methods for producing primers are known and include for example chemical synthesis.

In certain embodiments, a primer has a size of at least 15 bases, at least 16 bases, at least 17 bases, at least 18 bases, at least 19 bases, at least 20 bases, at least 21 bases, at least 22 bases, at least 23 bases, at least 24 bases, or at least 25 bases. Other primer sizes are contemplated.

In certain embodiments, a primer comprises a nucleotide sequence that is not a naturally occurring nucleotide sequence (a non-naturally occurring sequence) or does not hybridize to a naturally occurring sequence under stringent conditions. Naturally occurring nucleotide sequences are as described herein. In certain embodiments, a primer comprises a nucleotide sequence that is not able to substantially prime synthesis by a polymerase from a naturally occurring nucleotide sequence. In certain embodiments, a primer comprises a nucleotide sequence that is not able to substantially prime synthesis by a polymerase from a naturally occurring nucleotide sequence under stringent conditions. In certain embodiments, a primer has a sequence that is not a naturally occurring genomic nucleotide sequence. In certain embodiments, a primer comprises a sequence that does not hybridize to a naturally occurring genomic nucleotide sequence under stringent conditions. In certain embodiments, a primer comprises a nucleotide sequence that is not able to substantially prime synthesis by a polymerase from a naturally occurring genomic nucleotide sequence under stringent conditions.

Stringent conditions are as described, for example, in Sambrook et al. Molecular Cloning: A Laboratory Manual. 3rd ed. Cold Spring Harbor Laboratory Press, New York, N.Y. 2000. Methods for screening a sequence for the level of nucleotide sequence identity to other nucleotide sequences are know, and include for example the Basic Local Alignment Search Tool (BLAST). Methods for determining whether one nucleic acid may hybridize to another nucleic acid, or is able to substantially prime synthesis by a polymerase, are known.

In certain embodiments, a primer comprises a nucleotide sequence that is not present in a naturally occurring nucleotide sequence and the primer further comprises a 3′ base that is not present in the naturally occurring nucleotide sequence, thereby preventing amplification from a naturally occurring nucleotide sequence.

In certain embodiments, the primer comprises a nucleotide sequence that is not present in the genome of an organism to be tagged or screened. In certain embodiments, the primer comprises a nucleotide sequence that is not present in the genome of an organism to be tagged or screened. Examples of organisms are as described herein. For example, in certain embodiments a primer for use in identifying a human that is tagged may have a nucleotide sequence that is not present in the human genome.

In certain embodiments, a primer comprises a low level of nucleotide sequence identity with a naturally occurring nucleotide sequence. In certain embodiments, a primer comprises a low level of nucleotide sequence identity with a naturally occurring genomic nucleotide sequence.

In certain embodiments, a primer comprises a nucleotide sequence that is not identical with a naturally occurring nucleotide sequence. In certain embodiments, a primer comprises a nucleotide sequence that is not identical with a naturally occurring genomic nucleotide sequence. In certain embodiments, the genomic sequence is a human genomic sequence, an animal genomic sequence, a genomic sequence from a microorganism such as a bacterium, or any one or more of the aforementioned.

In certain embodiments, the genomic sequence is a human genomic sequence.

In certain embodiments, a primer comprises a sequence identity of 90% or less, 85% or less, 80% or less, 75% or less, 70% or less, or 65% or less with a naturally occurring nucleotide sequence. In certain embodiments, a primer comprises a sequence identity of 90% or less, 85% or less, 80% or less, 75% or less, 70% or less, or 65% or less with a naturally occurring genomic nucleotide sequence. Other levels of sequence identity are contemplated and are as described herein.

In certain embodiments, a primer has a sequence identity over its entire nucleotide sequence of 90% or less, 85% or less, 80% or less, 75% or less, 70% or less, or 65% or less with a naturally occurring nucleotide sequence. In certain embodiments, a primer comprises a sequence identity over its entire nucleotide sequence of 90% or less, 85% or less, 80% or less, 75% or less, 70% or less, or 65% or less with a naturally occurring genomic nucleotide sequence.

In certain embodiments, a primer comprises a sequence with one or more of a low propensity for secondary structure formation, hairpin formation, and/or intra- and/or intermolecular priming Methods for determining the propensity for secondary structure formation, hairpin formation, and/or for intra- and/or intermolecular priming are known.

In certain embodiments, the amplification of the nucleic acids in the nucleic acid tag comprises at least two different primers. In certain embodiments, the amplification of the nucleic acids in the nucleic acid tag comprises two different primers. In certain embodiments, the at least two different primers have a Tm that is within 5° C., 4° C., 3° C., 2° C., or 1° C. of each other. In certain embodiments, the two different primers have a Tm that is within 5° C., 4° C., 3° C., 2° C., or 1° C. of each other. Methods for determining Tm are known.

In certain embodiments, the amplification of the nucleic acids in the nucleic acid tag comprises a first primer. In certain embodiments, the first primer binds to a first primer region.

In certain embodiments, the nucleotide sequence of the first primer comprises SEQ ID NO.1, the complement of SEQ ID NO.1 or a nucleotide sequence with greater than 80% sequence identity to either of the aforementioned nucleotide sequences. Other levels of sequence identity are contemplated and are as described herein. In certain embodiments, the nucleotide sequence of the first primer is SEQ ID NO.1, the complement of SEQ ID NO.1 or a nucleotide sequence with greater than 80% sequence identity to either of the aforementioned nucleotide sequences. The nucleotide sequence of SEQ ID NO.1 is as follows: 5′ CCGCTGACAAGTAAACGCGATTGAT 3′ (SEQ ID NO.1).

In certain embodiments, the nucleotide sequence of the first primer comprises the following nucleotide sequence:

5′ NNGCTGACAAGTAAACGCGATTGNN 3′ (SEQ ID NO.30), wherein N is A, C, G, T or no nucleotide, or the complement of SEQ ID NO.30.

In certain embodiments, the nucleotide sequence of the first primer comprises a nucleotide sequence selected from one of the following nucleotide sequences, or the complement of one of the following nucleotide sequences:

(SEQ ID NO. 1)
5′ CCGCTGACAAGTAAACGCGATTGAT 3′;
wherein N is A, C, G, T
(SEQ ID NO. 30)
5′ NNGCTGACAAGTAAACGCGATTGNN 3′,
or
no nucleotide;
(SEQ ID NO. 31)
5′ CCGCTGACAAGTAAACGCGATTGAT 3′;
(SEQ ID NO. 32)
5′ CGCTGACAAGTAAACGCGATTGAT 3′;
(SEQ ID NO. 33)
5′ GCTGACAAGTAAACGCGATTGAT 3′;
(SEQ ID NO. 34)
5′ CCGCTGACAAGTAAACGCGATTGA 3′;
(SEQ ID NO. 35)
5′ CCGCTGACAAGTAAACGCGATTG 3′;
(SEQ ID NO. 36)
5′ CGCTGACAAGTAAACGCGATTGA 3′;
(SEQ ID NO. 37)
5′ CGCTGACAAGTAAACGCGATTG 3′;
(SEQ ID NO. 38)
5′ GCTGACAAGTAAACGCGATTGA 3′;
(SEQ ID NO. 39)
5′ GCTGACAAGTAAACGCGATTGA 3′;
(SEQ ID NO. 40)
5′ GCTGACAAGTAAACGCGATTG 3′;
(SEQ ID NO. 41)
5′ (G/C)(G/C)GCTGACAAGTAAACGCGATTG(A/T)(A/T) 3′;
(SEQ ID NO. 42)
5′ (G/C)GCTGACAAGTAAACGCGATTG(A/T)(A/T) 3′;
(SEQ ID NO. 43)
5′ GCTGACAAGTAAACGCGATTG(A/T)(A/T) 3′;
(SEQ ID NO. 44)
5′ (G/C)(G/C)GCTGACAAGTAAACGCGATTG(A/T) 3′;
and
(SEQ ID NO. 45)
5′ (G/C)(G/C)GCTGACAAGTAAACGCGATTG 3′;

In certain embodiments, one or more of the plurality of amplification products comprises one or more cleavage sites for a restriction endonuclease. In certain embodiments, the plurality of amplification products comprises one or more cleavage sites for a restriction endonuclease. Cleavage sites for restriction endonucleases are as described herein.

In certain embodiments, the amplification of the nucleic acids in the nucleic acid tag (and thereby the amplification of one or more of the plurality of selected different nucleic acids if present in the nucleic acid tag) comprises a second primer. In certain embodiments, the second primer binds to a second primer region.

In certain embodiments, the nucleotide sequence of the second primer comprises SEQ ID NO.2, the complement of SEQ ID NO.2 or a nucleotide sequence with greater than 80% sequence identity to either of the aforementioned nucleotide sequences. Other levels of sequence identity are contemplated and are as described herein. In certain embodiments, the nucleotide sequence of the second primer is SEQ ID NO.2, the complement of SEQ ID NO.2 or a nucleotide sequence with greater than 80% sequence identity to either of the aforementioned nucleotide sequences. The nucleotide sequence of SEQ ID NO.2 is as follows: 5′ TGCCTACTGAAAAGTCGGACCATAG 3′ (SEQ ID NO.2).

In certain embodiments, the nucleotide sequence of the second primer comprises the following nucleotide sequence:

5′ NNCCTACTGAAAAGTCGGACCATNN 3′ (SEQ ID NO.46), wherein N is A, C, G, T or no nucleotide, or the complement of SEQ ID NO.46.

In certain embodiments, the nucleotide sequence of the second primer comprises a nucleotide sequence selected from one of following nucleotide sequences, or the complement of one of the following nucleotide sequences:

(SED ID NO. 2)
5′ TGCCTACTGAAAAGTCGGACCATAG 3′;
wherein N is A, C, G, T
(SEQ ID NO. 46)
5′ NNCCTACTGAAAAGTCGGACCATNN 3′,
or
no nucleotide,
or
the complement of SEQ ID NO. 46.
(SED ID NO. 47)
5′ GCCTACTGAAAAGTCGGACCATAG 3′;
(SED ID NO. 48)
5′ CCTACTGAAAAGTCGGACCATAG 3′;
(SED ID NO. 49)
5′ TGCCTACTGAAAAGTCGGACCATA 3′;
(SED ID NO. 50)
5′ TGCCTACTGAAAAGTCGGACCAT 3′;
(SED ID NO. 51)
5′ GCCTACTGAAAAGTCGGACCATA 3′;
(SED ID NO. 52)
5′ GCCTACTGAAAAGTCGGACCAT 3′;
(SED ID NO. 53)
5′ GCCTACTGAAAAGTCGGACCAT 3′;
(SED ID NO. 54)
5′ CCTACTGAAAAGTCGGACCATA 3′;
(SED ID NO. 55)
5′ CCTACTGAAAAGTCGGACCAT 3′;
(SEQ ID NO. 56)
5′ (T/A)(G/C)CCTACTGAAAAGTCGGACCAT(A/T)(G/C) 3′;
(SEQ ID NO. 57)
5′ (G/C)CCTACTGAAAAGTCGGACCAT(A/T)(G/C) 3′;
(SEQ ID NO. 58)
5′ CCTACTGAAAAGTCGGACCAT(A/T)(G/C) 3′;
(SEQ ID NO. 59)
5′ (T/A)(G/C)CCTACTGAAAAGTCGGACCAT(A/T) 3′
and
(SEQ ID NO. 60)
5′ (T/A)(G/C)CCTACTGAAAAGTCGGACCAT 3′.

In certain embodiments, the amplifying of the nucleic acids in the nucleic acid tag (and thereby the amplification of one or more of the plurality of selected different nucleic acids if present in the nucleic acid tag) comprises a first primer and/or a second primer. In certain embodiments, the amplifying of the nucleic acids in the nucleic acid tag (and thereby the amplification of one or more of the plurality of selected different nucleic acids if present in the nucleic acid tag) comprises a first primer and a second primer.

In certain embodiments, the method comprises amplification comprising a primer to the first primer region and a primer to the second primer region.

In certain embodiments, the nucleotide sequence of the first primer and/or the nucleotide sequence of the second primer comprise a sequence identity of 80% or less with a naturally occurring nucleotide sequence. Other levels of sequence identity are contemplated and are as described herein. In certain embodiments, the nucleotide sequence of the first primer and/or the nucleotide sequence of the second primer comprise a sequence identity of 80% or less with a naturally occurring genomic nucleotide sequence. Other levels of sequence identity are contemplated and are as described herein.

In certain embodiments, the amplification comprises a first primer and/or a second primer, the nucleotide sequence of the first primer and/or the nucleotide sequence of the second primer comprising a sequence identity of 80% or less with a naturally occurring nucleotide sequence. In certain embodiments, the amplification comprises a first primer and/or a second primer, the nucleotide sequence of the first primer and/or the nucleotide sequence of the second primer comprising a sequence identity of 80% or less with a naturally occurring genomic nucleotide sequence. Other levels of sequence identity are contemplated and are as described herein.

In certain embodiments, the method comprises cleaving one or more of the plurality of amplification products with one or more restriction endonucleases. In certain embodiments, the method comprises cleaving the plurality of amplification products with one or more restriction endonucleases. Methods for cleaving nucleic acids with restriction endonucleases are known.

In certain embodiments, the detecting of one or more of the selected different nucleic acids in the nucleic acid tag comprises determination of the size of the amplification products. In certain embodiments, the detecting of the selected different nucleic acids in the nucleic acid tag comprises determination of the size of the amplification products. In certain embodiments, the detecting of the plurality of selected different nucleic acids in the nucleic acid tag comprises determination of the size of the amplification products. Methods for determining the size of nucleic acids are known, and include, for example, electrophoretic separation and/or mass separation.

In certain embodiments, the method comprises determining the presence of the selected different nucleic acids by the size of the amplification products. In certain embodiments, the method comprises determining the presence of the plurality of selected different nucleic acids by the size of the amplification products.

In certain embodiments, the detecting of the selected different nucleic acids comprises electrophoretic separation of one or more of the amplification products. In certain embodiments, the detecting of the plurality of selected different nucleic acids comprises electrophoretic separation of one or more of the amplification products. In certain embodiments, the detecting of the plurality of selected different nucleic acids comprises electrophoretic separation of the plurality of amplification products. Methods for electrophoretic separation are known.

In certain embodiments, the electrophoretic separation comprises capillary electrophoresis, such as capillary gel electrophoresis. In certain embodiments, the electrophoretic separation comprises gel electrophoresis. Methods for performing capillary electrophoresis and/or gel electrophoresis, including capillary gel electrophoresis, are known.

In certain embodiments, the detecting of one or more of the plurality of selected different nucleic acids comprises mass separation of one or more of the amplification products. In certain embodiments, the detecting of the selected different nucleic acids comprises mass separation of the plurality of amplification products. In certain embodiments, the detecting of the plurality of selected different nucleic acids comprises mass separation of the plurality of amplification products. In certain embodiments, the mass separation comprises mass spectrometry. Methods for performing mass separation, including mass spectrometry, are known.

In certain embodiments, the methods of the present disclosure comprise comparing the size of one or more amplification products with one or more reference nucleic acids. In certain embodiments, the methods of the present disclosure comprise comparing the size of one or more of the plurality of amplification products with one or more reference nucleic acids. In certain embodiments, the methods of the present disclosure comprise comparing the size of the plurality of amplification products with one or more reference nucleic acids. In certain embodiments, the detecting of one or more of the selected different nucleic acids comprises labelling of one or more of the amplification products.

In certain embodiments, the detecting of one or more of the plurality of selected different nucleic acids comprises labelling of one or more of the plurality of amplification products. In certain embodiments, the detecting of the plurality of selected different nucleic acids comprises labelling of the plurality of amplification products. Methods for labelling nucleic acids are known. In certain embodiments, the labelling comprises labelling with a fluorescent tag. Fluorescent tags are known and available commercially.

In certain embodiments, the labelling comprises amplification with a fluorescently labelled primer. In certain embodiments, the labelling comprises amplification with a fluorescently labelled nucleotide.

In certain embodiments, the detecting of one or more of the selected different nucleic acids in the nucleic acid tag comprises comparing the size of one or more amplification products with one or more reference nucleic acids. In certain embodiments, the detecting of the selected different nucleic acids in the nucleic acid tag comprises comparing the size of one or more of the amplification products with one or more reference nucleic acids. In certain embodiments, the detecting of the plurality of selected different nucleic acids in the nucleic acid tag comprises comparing the size of one or more amplification products with one or more reference nucleic acids. In certain embodiments, the detecting of the selected different nucleic acids in the nucleic acid tag comprises comparing the size of the plurality of amplification products with one or more reference nucleic acids. In certain embodiments, the detecting the plurality of selected different nucleic acids in the nucleic acid tag comprises comparing the size of the plurality of amplification products with one or more reference nucleic acids. Reference nucleic acids are known, and include for example reference nucleic acids of known size and/or reference nucleic acids of known mass and/or nucleotide sequence.

In certain embodiments, the methods of the present disclosure comprise determining the ratio of one or more of the plurality of selected different nucleic acids to one or more other nucleic acids. In certain embodiments, the methods of the present disclosure comprise determining the ratio of the selected different nucleic acids to one or more other nucleic acids. In certain embodiments, the methods of the present disclosure comprise determining the ratio of the plurality of selected different nucleic acids to one or more other nucleic acids. In certain embodiments, this may also be used to identify, or assist with identifying, the object marked with the nucleic acid tag. In certain embodiments, the ratio is different between specific nucleic acid tags. Methods for determining the concentration and/or ratio of nucleic acids are known.

In certain embodiments, the method comprises determining the ratio of one or more of the plurality of selected different nucleic acids to one or more other selected different nucleic acids.

In certain embodiments, the identifying of the object comprises determining the ratio of one or more of the plurality of selected different nucleic acids to one or more other nucleic acids. In certain embodiments, this may also be used to identify the object marked with the nucleic acid tag. In certain embodiments, the ratio is different between specific nucleic acid tags. In certain embodiments, the ratio is substantially the same between specific nucleic acid tags. In certain embodiments, the identifying of the object comprises determining the ratio of the plurality of selected different nucleic acids to one or more other nucleic acids. In certain embodiments, the identifying of the object comprises determining the ratio of one or more of the plurality of selected different nucleic acids to one or more other selected different nucleic acids.

In certain embodiments, the detection of the selected different nucleic acid comprises serial and/or parallel detection of one or more of the selected different nucleic acids. In certain embodiments, the detection of the plurality of selected different nucleic acid comprises serial and/or parallel detection of one or more of the selected different nucleic acids. In certain embodiments, the detection of the plurality of selected different nucleic acid comprises serial and/or parallel detection of the plurality of selected different nucleic acids.

In certain embodiments, the detection of the selected different nucleic acid comprises multiplex detection of one or more of the selected different nucleic acids. In certain embodiments, the detection of the plurality of selected different nucleic acid comprises multiplex detection of one or more of the selected different nucleic acids. In certain embodiments, the detection of the plurality of selected different nucleic acid comprises multiplex detection of the plurality of selected different nucleic acids.

In certain embodiments, the methods of the present disclosure comprise serial or parallel detection of one or more of the selected different nucleic acids. In certain embodiments, the methods of the present disclosure comprise serial or parallel detection of the selected different nucleic acids. In certain embodiments, the methods of the present disclosure comprise serial or parallel detection of the plurality of selected different nucleic acids.

In certain embodiments, the methods of the present disclosure comprise multiplex detection of one or more of the selected different nucleic acids. In certain embodiments, the methods of the present disclosure comprise multiplex detection of the selected different nucleic acids. In certain embodiments, the methods of the present disclosure comprise multiplex detection of the plurality of selected different nucleic acids.

In certain embodiments, detecting of nucleic acids comprises fluorescent detection. In certain embodiments, detecting of nucleic acids comprises mass spectrometry. Other methods of detecting are contemplated, such as detection by hybridization with a probe, or detection utilising radioactive nucleotides incorporated into amplification products, and/or detection with nucleic acid intercalating agents.

In certain embodiments, the detecting of the selected different nucleic acids comprises production of an electrophoretic pattern of the size of the amplified products. In certain embodiments, the detecting of the plurality of selected different nucleic acids comprises production of an electrophoretic pattern of the size of the amplified products. In certain embodiments, the detecting of the selected different nucleic acids comprises production of a mass spray spectrogram of the size of the amplified products. In certain embodiments, the detecting of the plurality of selected different nucleic acids comprises production of a mass spray spectrogram of the size of the amplified products. Other methods are contemplated.

In certain embodiments, the detecting of the selected different nucleic acids comprises fingerprint or DNA profiling analysis. In certain embodiments, the detecting of the plurality of selected different nucleic acids comprises fingerprint or DNA profiling analysis. Methods for fingerprinting and DNA profiling are known.

In certain embodiments, the methods comprise detection of one or more of the selected different nucleic acids at a concentration of 1000 pg/ml or less, 500 pg/ml or less, 100 pg/ml or less, 50 pg/ml or less, 10 pg/ml or less, 5 pg/ml or less, 1 pg/ml or less, 500 fg/ml or less, 100 fg/ml or less, 50 fg/ml or less, 10 fg/ml or less, 5 fg/ml or less. In certain embodiments, the methods comprise detection of the different nucleic acids wherein each of the different nucleic acids has a concentration at one of the aforementioned concentrations. In certain embodiments, the methods comprise detection of the different nucleic acids wherein all the different nucleic acids have a total concentration at one of the aforementioned concentrations.

In certain embodiments, the method comprises detection of the selected different nucleic acids at a concentration of 1000 pg/ml or less, 500 pg/ml or less, 100 pg/ml or less, 50 pg/ml or less, 10 pg/ml or less, 5 pg/ml or less, 1 pg/ml or less, 500 fg/ml or less, 100 fg/ml or less, 50 fg/ml or less, 10 fg/ml or less, 5 fg/ml or less. In certain embodiments, the method comprises detection of the plurality of selected different nucleic acids at a concentration of 1000 pg/ml or less, 500 pg/ml or less, 100 pg/ml or less, 50 pg/ml or less, 10 pg/ml or less, 5 pg/ml or less, 1 pg/ml or less, 500 fg/ml or less, 100 fg/ml or less, 50 fg/ml or less, 10 fg/ml or less, 5 fg/ml or less.

In certain embodiments, the nucleic acid tag comprises an amount of one or more of the plurality of selected different nucleic acids of 2000 ng or less, 1000 ng or less, 900 ng or less, 800 ng or less, 700 ng or less, 600 ng or less, 500 ng or less, 400 ng or less, 300 ng or less, 200 ng or less, 100 ng or less, 50 ng or less, 40 ng or less, 30 ng or less, 20 ng or less, 10 ng or less, 5 ng or less, 1 ng/less, 500 pg or less, 100 pg or less, 50 pg or less, 10 pg or less, 5 pg or less, or 1 pg or less.

In certain embodiments, the nucleic acid tag comprises an amount of the plurality of selected different nucleic acids of 2000 ng or less, 1000 ng or less, 900 ng or less, 800 ng or less, 700 ng or less, 600 ng or less, 500 ng or less, 400 ng or less, 300 ng or less, 200 ng or less, 100 ng or less, 50 ng or less, 40 ng or less, 30 ng or less, 20 ng or less, 10 ng or less, 5 ng or less, 1 ng/less, 500 pg or less, 100 pg or less, 50 pg or less, 10 pg or less, 5 pg or less, or 1 pg or less.

In certain embodiments, the nucleic acid tag comprises an amount of one or more of the plurality of selected different nucleic acids of 2000 ng or greater, 1000 ng or greater, 900 ng or greater, 800 ng or greater, 700 ng or greater, 600 ng or greater, 500 ng or greater, 400 ng or greater, 300 ng or greater, 200 ng or greater, 100 ng or greater, 50 ng or greater, 40 ng or greater, 30 ng or greater, 20 ng or greater, 10 ng or greater, 5 ng or greater, 1 ng/greater, 500 pg or greater, 100 pg or greater, 50 pg or greater, 10 pg or greater, 5 pg or greater, or 1 pg or greater. In certain embodiments, the methods comprise detection of the different nucleic acids wherein each of the different nucleic acids has a concentration at one of the aforementioned concentrations. In certain embodiments, the methods comprise detection of the different nucleic acids wherein all the different nucleic acids have a total concentration at one of the aforementioned concentrations.

In certain embodiments, the nucleic acid tag comprises an aforementioned amount for each of the one or more different nucleic acids. In certain embodiments, the nucleic acid tag comprises an amount of the plurality of selected different nucleic acids of 2000 ng or greater, 1000 ng or greater, 900 ng or greater, 800 ng or greater, 700 ng or greater, 600 ng or greater, 500 ng or greater, 400 ng or greater, 300 ng or greater, 200 ng or greater, 100 ng or greater, 50 ng or greater, 40 ng or greater, 30 ng or greater, 20 ng or greater, 10 ng or greater, 5 ng or greater, 1 ng/greater, 500 pg or greater, 100 pg or greater, 50 pg or greater, 10 pg or greater, 5 pg or greater, or 1 pg or greater.

In certain embodiments, the nucleic acid tag comprises an amount of one or more of the plurality of selected different nucleic acids of 1-10 ng, 1-20 ng, 1-30 ng, 1-40 ng, 1-50 ng, 1-100 ng, 1-200 ng, 1-300 ng, 1-400 ng, 1-500 ng, 1-1000 ng, 10-20 ng, 10-30 ng, 10-40 ng, 10-50 ng, 10-100 ng, 10-200 ng, 10-300 ng, 10-400 ng, 10-500 ng, 10-1000 ng, 20-30 ng, 20-40 ng, 20-50 ng, 20-100 ng, 20-200 ng, 20-300 ng, 20-400 ng, 20-500 ng, 20-1000 ng, 30-40 ng, 30-50 ng, 30-100 ng, 30-200 ng, 30-300 ng, 30-400 ng, 30-500 ng, 30-1000 ng, 40-50 ng, 40-100 ng, 40-200 ng, 40-300 ng, 40-400 ng, 40-500 ng, 40-1000 ng, 50-100 ng, 50-200 ng, 50-300 ng, 50-400 ng, 50-500 ng, 50-1000 ng, 100-200 ng, 100-300 ng, 100-400 ng, 100-500 ng, 100-1000 ng, 200-300 ng, 200-400 ng, 200-500 ng, 200-1000 ng, 300-400 ng, 300-500 ng, 300-1000 ng 400-500 ng, 400-1000 ng, or 500-1000 ng. In certain embodiments, the methods comprise detection of the different nucleic acids wherein each of the different nucleic acids has a concentration at one of the aforementioned concentrations. In certain embodiments, the methods comprise detection of the different nucleic acids wherein all the different nucleic acids have a total concentration at one of the aforementioned concentrations.

In certain embodiments, the nucleic acid tag comprises an amount of the plurality of selected different nucleic acids of 1-10 ng, 1-20 ng, 1-30 ng, 1-40 ng, 1-50 ng, 1-100 ng, 1-200 ng, 1-300 ng, 1-400 ng, 1-500 ng, 1-1000 ng, 10-20 ng, 10-30 ng, 10-40 ng, 10-50 ng, 10-100 ng, 10-200 ng, 10-300 ng, 10-400 ng, 10-500 ng, 10-1000 ng, 20-30 ng, 20-40 ng, 20-50 ng, 20-100 ng, 20-200 ng, 20-300 ng, 20-400 ng, 20-500 ng, 20-1000 ng, 30-40 ng, 30-50 ng, 30-100 ng, 30-200 ng, 30-300 ng, 30-400 ng, 30-500 ng, 30-1000 ng, 40-50 ng, 40-100 ng, 40-200 ng, 40-300 ng, 40-400 ng, 40-500 ng, 40-1000 ng, 50-100 ng, 50-200 ng, 50-300 ng, 50-400 ng, 50-500 ng, 50-1000 ng, 100-200 ng, 100-300 ng, 100-400 ng, 100-500 ng, 100-1000 ng, 200-300 ng, 200-400 ng, 200-500 ng, 200-1000 ng, 300-400 ng, 300-500 ng, 300-1000 ng 400-500 ng, 400-1000 ng, or 500-1000 ng.

In certain embodiments, the nucleic acid tag comprises an amount of one or more of the plurality of selected different nucleic acids of 10-400 ng. In certain embodiments, the nucleic acid tag comprises an aforementioned amount for each of the one or more different nucleic acids. In certain embodiments, the nucleic acid tag comprises an amount of the plurality of selected different nucleic acids of 10-400 ng.

In certain embodiments, the identifying of the object by the presence of the plurality of selected different nucleic acids comprises determination of the presence of the respective sizes of the amplification products produced. For example, the presence of a specific pattern of amplification products is indicative of the presence of specific selected different nucleic acids in the nucleic acid tag, and which thereby allows identification of the object by correlating the pattern of specific different nucleic acids with a nucleic acid tag known to produce the same pattern of amplified products. Examples of specific patterns are as described herein, and include for example electrophoresis patterns and/or mass spray patterns.

In certain embodiments, the identifying of the object by the presence of plurality of selected different nucleic acids comprises determination of the presence of a specific ratio of one or more of the amplification products of different size produced. For example, the presence of a specific pattern of ratios of differently sized amplification products is indicative of the presence of specific selected different nucleic acids in the nucleic acid tag, and which thereby allows identification of the object by correlating the ratio of specific different nucleic acids with a nucleic acid tag known to have the same ratio of amplified products.

Certain embodiments of the present disclosure provide a method of identifying an object marked with a nucleic acid tag comprising a plurality of selected different nucleic acids, the method comprising:
sampling the object comprising the nucleic acid tag, wherein the plurality of selected different nucleic acids comprises a first primer region, a second primer region and an intervening region between the first primer region and the second primer region, the size of the intervening region being different between the selected different nucleic acids;
amplifying nucleic acids in the sample, the amplification comprising a primer to the first primer region and a primer to the second primer region;
determining the presence of the plurality of selected different nucleic acids by the size of the amplification products; and
identifying the object marked with the nucleic acid tag by the presence of the plurality of selected different nucleic acids in the nucleic acid tag.

In certain embodiments, the methods of identification described herein are used to determine whether an object has been marked with a nucleic acid tag, to identify a human or animal, to identify an object for forensic purposes, to identify a counterfeit product, to identify parallel trading of a product, to track an object, to identify the source of a product, for security marking, or to distinguish one product from another product.

Certain embodiments of the present disclosure also provide methods of determining whether an object has been marked with a nucleic acid tag, as described herein.

Certain embodiments of the present disclosure provide a method of determining whether an object has been marked with a nucleic acid tag, the nucleic acid tag comprising a plurality of selected different nucleic acids, as described herein.

Certain embodiments of the present disclosure provide a method of determining whether an object has been marked with a nucleic acid tag, the nucleic acid tag comprising a plurality of selected different nucleic acids, the method comprising:
amplifying nucleic acids from the object;
detecting the plurality of selected different nucleic acids in the nucleic acid tag by the size of the plurality of amplification products; and
determining whether the object has been marked with the nucleic acid tag by the presence of the plurality of selected different nucleic acids in the nucleic tag.

Amplification of nucleic acids is as described herein. Detection of nucleic acids is as described herein.

Certain embodiments of the present disclosure provide a method of determining whether an object has been marked with a nucleic acid tag, the nucleic acid tag comprising a plurality of selected different nucleic acids, the method comprising:
obtaining a sample from the object;
amplifying nucleic acids in the sample, wherein amplification of the plurality of selected different nucleic acids in the nucleic acid tag produces a plurality of amplification products with a different size;
detecting the plurality of selected different nucleic acids in the nucleic acid tag by the size of the plurality of amplification products; and
determining whether the object has been marked with the nucleic acid tag by the presence of the plurality of selected different nucleic acids in the nucleic tag.

Certain embodiments of the present disclosure also provide isolated nucleic acids and pluralities of isolated nucleic acids.

Certain embodiments of the present disclosure also provide vectors comprising the nucleic acids as described herein, and hosts comprising the vectors. Suitable vectors include vectors for use in prokaryotes or eukaryote hosts, which are known and/or commercially available. Certain embodiments of the present disclosure provide a host cell comprising a vector as described herein.

Certain embodiments of the present disclosure also provide methods for marking an object with a nucleic acid tag by adding to the object a nucleic acid, as described herein.

Certain embodiments of the present disclosure provide a method of marking or tagging an object, the method using an isolated nucleic acid as described herein. In certain embodiments, the object is marked or tagged with a single isolated nucleic acid as described herein. In certain embodiments, the object is marked or tagged with a plurality of isolated nucleic acids as described herein.

The term “isolated” refers to a molecule, for example a nucleic acid, that is at least partially separated from other molecules. For example, the term “isolated” may refer to a nucleic acid molecule, for example an oligonucleotide or a polynucleotide, that is at least partially separated from other nucleic acid molecules that are present in a source of the nucleic acid.

Examples of isolated nucleic acid molecules include recombinant DNA molecules contained in a vector, recombinant DNA molecules maintained in a heterologous host cell, partially or substantially purified nucleic acid molecules, nucleic acids produced by amplification, and synthetic DNA or RNA molecules. An isolated nucleic acid may be substantially or partially free of other cellular material (including other nucleic acids) or culture medium, for example when produced by recombinant techniques. Methods for producing isolated nucleic acids are known, including recombinant DNA technology or chemical synthesis, for example as described in Sambrook et al. Molecular Cloning: A Laboratory Manual. 3rd ed. Cold Spring Harbor Laboratory Press, New York, N.Y. 2000 or Current Protocols in Molecular Biology. Ed. Ausubel et al. John Wiley & Sons, Inc. Cambridge, Mass., 2000.

Certain embodiments of the present disclosure provide isolated nucleic acids or a plurality of isolated nucleic acids. Certain embodiments of the present disclosure provide a vector comprising a nucleic acid as described herein. Certain embodiments of the present disclosure provide a host cell comprising a vector as described herein.

The term “vector” is also intended to encompass other vectors, such as phages, viruses (such as SV40, CMV), baculovirus, adenovirus, transposons, IS elements, phasmids, phagemids, cosmids, linear or circular DNA and RNA. Vector nucleic acid may be introduced into prokaryotic or eukaryotic cells by a known method, including via conventional transformation or transfection techniques such as calcium phosphate or calcium chloride coprecipitation, DEAE-dextran-mediated transfection, lipofection, natural competence, chemically mediated transfer, electroporation or particle bombardment. Suitable methods for the transformation or transfection of host cells, are as described for example in Sambrook et al. (Molecular Cloning: A Laboratory Manual., 3rd Ed., Cold Spring Harbor Laboratory, Cold Spring Harbor Laboratory Press, Cold Spring Harbor, N.Y., 2000).

In certain embodiments, the isolated nucleic acid comprises a size of at least 20 base pairs, at least 25 base pairs, at least 30 base pairs, at least 40 base pairs, at least 50 base pairs, at least 100 base pairs, at least 150 base pairs, at least 200 base pairs, at least 215 base pairs, at least 240 base pairs, at least 250 base pairs, at least 300 base pairs, at least 400 base pairs or at least 500 base pairs. Other sizes are contemplated.

In certain embodiments, the isolated nucleic acid comprise a size of 25 base pairs or less, 30 base pairs or less, 40 base pairs or less, 50 base pairs or less, 100 base pairs or less, 150 base pairs or less, 200 base pairs or less, 215 base pairs or less, 240 base pairs or less, 250 base pairs or less, 300 base pairs or less, 400 base pairs or less, or 500 base pairs or less.

In certain embodiments, the isolated nucleic acid comprise a size of 20-500, 20-400, 20-300, 20-250, 20-240, 20-215, 20-200, 20-150, 20-100, 20-50, 20-40, 20-30, 20-25, 25-500, 25-400, 25-300, 25-250, 25-240, 25-215, 25-200, 25-150, 25-100, 25-50, 25-40, 25-30, 30-500, 30-400, 30-300, 30-250, 30-240, 30-215, 30-200, 30-150, 30-100, 30-50, 30-40, 40-500, 40-400, 40-300, 40-250, 40-240, 40-215, 40-200, 40-150, 40-100, 40-50, 50-500, 50-400, 50-300, 50-250, 50-240, 50-215, 50-200, 50-150, 50-100, 10-500, 100-400, 100-300, 100-250, 100-240, 100-215, 100-200, 100-150, 150-500, 150-400, 150-300, 150-250, 150-240, 150-215, 150-200, 200-500, 200-400, 200-300, 200-250, 200-240, 200-215, 215-500, 215-400, 215-300, 215-250, 215-240, 240-500, 240-400, 240-300, 240-250, 250-500, 250-400, 250-300, 300-500, 300-400, or 400-500 base pairs.

In certain embodiments, the isolated nucleic acids have a size of less than 500 base pairs. In certain embodiments, the isolated nucleic acids have a size in the range from 100 to 300 base pairs.

Certain embodiments of the present disclosure provide an isolated nucleic acid comprising: (i) SEQ ID NO.1 and/or the complement thereof; and/or (ii) a nucleotide sequence with greater than 80% sequence identity to SEQ ID NO.1 or the complement thereof. Other levels of sequence identity are contemplated and are as described herein.

Certain embodiments of the present disclosure provide an isolated nucleic acid comprising: (i) SEQ ID NO.2 and/or the complement thereof; and/or (ii) a nucleotide sequence with greater than 80% sequence identity to SEQ ID NO.2 or the complement thereof. Other levels of sequence identity are contemplated and are as described herein.

Certain embodiments of the present disclosure provide an isolated nucleic acid comprising: (i) SEQ ID NO.1 and SEQ ID NO.2, and/or the complement of either or both of the aforementioned nucleotide sequences, and/or a nucleotide sequence with greater than 80% sequence identity to either or both of the aforementioned nucleotide sequences or the complement thereof. Other levels of sequence identity are contemplated and are as described herein.

Certain embodiments of the present disclosure provide an isolated nucleic acid comprising: (i) SEQ ID NO.3 and/or the complement thereof; and/or (ii) a nucleotide sequence with greater than 80% sequence identity to SEQ ID NO.3 or the complement thereof. Other levels of sequence identity are contemplated and are as described herein.

Certain embodiments of the present disclosure provide an isolated nucleic acid comprising: (i) SEQ ID NO.4 and/or the complement thereof; and/or (ii) a nucleotide sequence with greater than 80% sequence identity to SEQ ID NO.4 or the complement thereof. Other levels of sequence identity are contemplated and are as described herein.

Certain embodiments of the present disclosure provide an isolated nucleic acid comprising one or more of the following nucleotide sequences:
(i) SEQ ID NO.1 and/or the complement thereof, and/or a nucleotide sequence with greater than 80% sequence identity to SEQ ID NO.1 or the complement thereof;
(ii) SEQ ID NO.2 and/or the complement thereof, and/or a nucleotide sequence with greater than 80% sequence identity to SEQ ID NO.2 or the complement thereof;
(iii) SEQ ID NO.3 and/or the complement thereof, and/or a nucleotide sequence with greater than 80% sequence identity to SEQ ID NO.3 or the complement thereof;
(iv) SEQ ID NO.4 and/or the complement thereof, and/or a nucleotide sequence with greater than 80% sequence identity to SEQ ID NO.4 or the complement thereof; and
(v) one or more of SEQ NOs. 30 to 60, and/or the complement thereof, and/or a nucleotide sequence with greater than 80% sequence identity to one or more of SEQ ID NOs. 30 to 60 or the complement thereof.

Certain embodiments of the present disclosure provide an isolated nucleic acid comprising one or more of a first primer region, a second region, an intervening region and a region of shared sequence, as described herein. In certain embodiments, the intervening region has a size of at least 15 base pairs.

Certain embodiments of the present disclosure provide an isolated nucleic acid comprising:
a first primer region and/or a second primer region, the first primer region and/or the second primer region comprising a sequence identity of 80% or less with a naturally occurring sequence; and
an intervening region between the first primer region and the second primer region, the intervening region having a size of at least 15 base pairs.

In certain embodiments, the nucleotide sequence of the first primer region and the nucleotide sequence of the second primer region are different. In certain embodiments, the isolated nucleic acid comprises a cleavage site for a restriction endonuclease between the first primer region and the second primer region.

In certain embodiments, the nucleotide sequence of the intervening region comprises SEQ ID NO. 3 and/or the complement of SEQ ID NO.3.

In certain embodiments, the nucleotide sequence of the first primer region comprises SEQ ID NO.1, the complement of SEQ ID NO.1 or a nucleotide sequence with greater than 80% sequence identity to either of the aforementioned. In certain embodiments, the nucleotide sequence of the second primer region comprises SEQ ID NO.2, the complement of SEQ ID NO.2 or a nucleotide sequence with greater than 80% sequence identity to either of the aforementioned.

Certain embodiments of the present disclosure provide a plurality of isolated different nucleic acids, as described herein.

Certain embodiments of the present disclosure provide a plurality of isolated different nucleic acids, the plurality of isolated different nucleic acids comprising a first primer region and a second primer region, as described herein.

Certain embodiments of the present disclosure provide a plurality of isolated different nucleic acids, the plurality of isolated different nucleic acids comprising a first primer region and a second primer region, the first primer region and/or the second primer region comprising a sequence identity of 80% or less with a naturally occurring sequence.

Certain embodiments of the present disclosure provide a plurality of isolated different nucleic acids, the plurality of isolated different nucleic acids comprising an intervening region between a first primer region and a second primer region, as described herein.

Certain embodiments of the present disclosure provide a plurality of isolated different nucleic acids, the plurality of isolated different nucleic acids comprising an intervening region between a first primer region and a second primer region, wherein the size of the intervening region is different between the nucleic acids.

Certain embodiments of the present disclosure provide a plurality of isolated different nucleic acids, the plurality of isolated different nucleic acids comprising:
a first primer region and/or a second primer region, the first primer region and/or the second primer region comprising a sequence identity of 80% or less with a naturally occurring sequence; and
an intervening region between the first primer region and the second primer region, wherein the size of the intervening region is different between the plurality of different nucleic acids.

Certain embodiments of the present disclosure provide a plurality of isolated different nucleic acids, the plurality of different nucleic acids comprising:
a first primer region and a second primer region, wherein the nucleotide sequence of the first primer region and/or the nucleotide sequence of the second primer region comprise a sequence identity of 80% or less with a naturally occurring genomic nucleotide sequence; and
an intervening region between the first primer region and the second primer region; wherein the size of the intervening region is different between the different nucleic acids.

In certain embodiments, the plurality of isolated different nucleic acids comprises at least two different nucleic acids. In certain embodiments, the plurality of isolated nucleic acids comprises at least two different nucleic acids. In certain embodiments, the plurality of isolated nucleic acids comprises three, four, five, six, or more different nucleic acids. In certain embodiments, the plurality of isolated nucleic acids comprises at least three, at least four, at least five, or at least six different nucleic acids.

In certain embodiments, one or more of the isolated different nucleic acids have a size of 500 base pairs or less. In certain embodiments, the plurality of isolated different nucleic acids have a size of 500 base pairs or less. In certain embodiments, one or more of the isolated different nucleic acids have a size of 400 base pairs or less. In certain embodiments, the plurality of isolated different nucleic acids have a size of 400 base pairs or less. In certain embodiments, one or more of the isolated different nucleic acids have a size of 300 base pairs or less. In certain embodiments, the plurality of isolated different nucleic acids have a size of 300 base pairs or less. In certain embodiments, one or more of the isolated different nucleic acids have a size of 200 base pairs or less. In certain embodiments, the plurality of isolated different nucleic acids have a size of 200 base pairs or less. In certain embodiments, one or more of the isolated different nucleic acids have a size of 100 base pairs or less. In certain embodiments, the plurality of isolated different nucleic acids have a size of 100 base pairs or less.

In certain embodiments, one or more of the plurality of isolated different nucleic acids have a size in the range from 100 to 500, 100 to 400, 100 to 300, 100 to 200, 200 to 500, 200 to 400, 200 to 300, 300 to 500, 300 to 400, or 400 to 500 base pairs. In certain embodiments, the plurality of isolated different nucleic acids have a size in the range from 100 to 500, 100 to 400, 100 to 300, 100 to 200, 200 to 500, 200 to 400, 200 to 300, 300 to 500, 300 to 400, or 400 to 500 base pairs.

In certain embodiments, one or more of the isolated different nucleic acids have a size in the range from 100 to 300 base pairs. In certain embodiments, the plurality of isolated different nucleic acids have a size in the range from 100 to 300 base pairs.

In certain embodiments, one or more of the isolated different nucleic acids differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, at least 1 base pair or less than 1 base pair. In certain embodiments, the plurality of isolated different nucleic acids differ in size by at least 50, at least 45, at least 40, at least 35, at least 30, at least 25, at least 20, at least 15, at least 10, at least 5 base pairs, at least 4 base pairs, at least 3 base pairs, at least 2 base pairs, at least 1 base pair, or less than 1 base pair.

In certain embodiments, the intervening region differs in size by at least 5 base pairs between the plurality of isolated different nucleic acids. In certain embodiments, the intervening region differs in size by at least 1 base pair between the plurality of isolated different nucleic acids.

In certain embodiments, the ratio of one or more of the plurality of isolated different nucleic acids to one or more other different nucleic acids is different.

In certain embodiments, the nucleotide sequence of the first primer region and/or the nucleotide sequence of the second primer region comprise a sequence identity of 80% or less with a naturally occurring nucleotide sequence. In certain embodiments, the nucleotide sequence of the first primer region and/or the nucleotide sequence of the second primer region comprise a sequence identity of 80% or less with a naturally occurring genomic nucleotide sequence.

In certain embodiments, the nucleotide sequence of the first primer region and the nucleotide sequence of the second primer region are different.

In certain embodiments, one or more of the plurality of isolated different nucleic acids comprises a cleavage site for a restriction endonuclease between the first primer region and the second primer region. In certain embodiments, the plurality of isolated different nucleic acids comprises a cleavage site for a restriction endonuclease between the first primer region and the second primer region.

In certain embodiments, one or more of the plurality of isolated different nucleic acids comprise a region of shared sequence between the first primer region and the second primer region. In certain embodiments, the plurality of isolated different nucleic acids comprise a region of shared sequence between the first primer region and the second primer region. In certain embodiments, the region of shared sequence comprises a cleavage site for a restriction endonuclease. In certain embodiments, the region of shared sequence has a length of 1 to 50 base pairs. In certain embodiments, the nucleotide sequence of the region of shared sequence comprises SEQ ID NO. 3 or the complement of SEQ ID NO.3

In certain embodiments, the nucleotide sequence of the first primer region comprises SEQ ID NO.1, the complement of SEQ ID NO.1 or a nucleotide sequence with greater than 80% sequence identity to either of the aforementioned nucleotide sequences. In certain embodiments, the nucleotide sequence of the first primer region comprises one or more of SEQ ID NOs. 30 to 45, the complement of one of the aforementioned nucleotide sequences, or a nucleotide sequence with greater than 80% sequence identity to one of the aforementioned nucleotide sequences.

In certain embodiments, the nucleotide sequence of the second primer region comprises SEQ ID NO.2, the complement of SEQ ID NO.2 or a nucleotide sequence with greater than 80% sequence identity to either of the aforementioned nucleotide sequences. In certain embodiments, the nucleotide sequence of the second primer region comprises one or more of SEQ ID NOs. 46 to 60, the complement of one of the aforementioned nucleotide sequences, or a nucleotide sequence with greater than 80% sequence identity to one of the aforementioned nucleotide sequences.

In certain embodiments, the nucleotide sequence of the first primer region and the nucleotide sequence of the second primer region are different.

In certain embodiments, the nucleotide sequence of the first primer region comprises a nucleotide sequence selected from (i) SEQ ID NO.1, the complement of SEQ ID NO.1 or a nucleotide sequence with greater than 80% sequence identity to either of the aforementioned nucleotide sequences, and/or (ii) a nucleotide sequence selected from one of SEQ ID NOs. 30 to 45 or the complement of one of the aforementioned nucleotide sequences.

In certain embodiments, the nucleotide sequence of the second primer region comprises a nucleotide sequence selected from (i) SEQ ID NO.2, the complement of SEQ ID NO.2 or a nucleotide sequence with greater than 80% sequence identity to either of the aforementioned nucleotide sequences, and/or (ii) a nucleotide sequence selected from one of SEQ ID NOs. 46 to 60 or the complement of one of the aforementioned nucleotide sequences.

In certain embodiments, the nucleotide sequence of the region of shared sequence comprises SEQ ID NO. 3, the complement of SEQ ID NO.3 or a nucleotide sequence with greater than 80% sequence identity to either of the aforementioned nucleotide sequences.

Certain embodiments of the present disclosure provide a marking composition comprising a nucleic acid, as described herein.

Certain embodiments of the present disclosure provide a method of marking an object with a nucleic acid tag, the method comprising adding a plurality of isolated different nucleic acids to the object, as described herein.

Certain embodiments of the present disclosure provide a marking composition, the composition comprising a plurality of isolated different nucleic acids, the plurality of isolated different nucleic acids comprising:
a first primer region and a second primer region, wherein the nucleotide sequence of the first primer region and/or the nucleotide sequence of the second primer region comprise a sequence identity of 80% or less with a naturally occurring nucleotide sequence; and
an intervening region between the first primer region and the second primer region; wherein the size of the intervening region is different between the selected different nucleic acids.

In certain embodiments, the marking composition comprises one or more non-nucleic acid agents. In certain embodiments, the marking composition comprises a buffer (for example Tris), a stabilizer, a colourant, a fluorescent agent, a detergent and/or a metal ion chelator (for example EDTA). In certain embodiments, the marking composition comprises a fluorescent agent. Other agents are contemplated.

In certain embodiments, the marking composition comprises a solid composition. In certain embodiments, the marking composition comprises a substantially solid composition. In certain embodiments, the marking composition comprises a powder, a dehydrate, or a lyophilised solid. In certain embodiments, the marking composition comprises a semi-solid composition. In certain embodiments, the marking composition comprises a gel. In certain embodiments, the marking composition comprises a liquid composition. In certain embodiments, the marking composition comprises a substantially liquid composition. In certain embodiments, the marking composition comprises an aqueous composition. In certain embodiments, the marking composition comprises a substantially aqueous composition. In certain embodiments, the marking composition comprises an ethanolic composition or a substantially ethanolic composition.

In certain embodiments, the marking composition comprises a sprayable composition. In certain embodiments, the marking composition is used an ink, a spray, or a paint.

In certain embodiments, the marking composition comprises a concentration of one or more of the plurality of isolated different nucleic acids of 2000 ng/ml or less, 1000 ng/ml or less, 900 ng/ml or less, 800 ng/ml or less, 700 ng/ml or less, 600 ng/ml or less, 500 ng/ml or less, 400 ng/ml or less, 300 ng/ml or less, 200 ng/ml or less, 100 ng/ml or less, 50 ng/ml or less, 40 ng/ml or less, 30 ng/ml or less, 20 ng/ml or less, 10 ng/ml or less, 5 ng/ml or less, 1 ng/ml/less, 500 pg/ml or less, 100 pg/ml or less, 50 pg/ml or less, 10 pg/ml or less, 5 pg/ml or less, or 1 pg/ml or less. In certain embodiments, the marking composition comprises a concentration of each of the different nucleic acids at a concentration at one of the aforementioned concentrations. In certain embodiments, the marking composition comprises a concentration of the different nucleic acids at a total concentration of one of the aforementioned concentrations.

In certain embodiments, the marking composition comprises a total concentration of the plurality of isolated different nucleic acids of 2000 ng/ml or less, 1000 ng/ml or less, 900 ng/ml or less, 800 ng/ml or less, 700 ng/ml or less, 600 ng/ml or less, 500 ng/ml or less, 400 ng/ml or less, 300 ng/ml or less, 200 ng/ml or less, 100 ng/ml or less, 50 ng/ml or less, 40 ng/ml or less, 30 ng/ml or less, 20 ng/ml or less, 10 ng/ml or less, 5 ng/ml or less, 1 ng/ml/less, 500 pg/ml or less, 100 pg/ml or less, 50 pg/ml or less, 10 pg/ml or less, 5 pg/ml or less, or 1 pg/ml or less.

In certain embodiments, the marking composition comprises a concentration of one or more of the plurality of isolated different nucleic acids of 2000 ng/ml or greater, 1000 ng/ml or greater, 900 ng/ml or greater, 800 ng/ml or greater, 700 ng/ml or greater, 600 ng/ml or greater, 500 ng/ml or greater, 400 ng/ml or greater, 300 ng/ml or greater, 200 ng/ml or greater, 100 ng/ml or greater, 50 ng/ml or greater, 40 ng/ml or greater, 30 ng/ml or greater, 20 ng/ml or greater, 10 ng/ml or greater, 5 ng/ml or greater, 1 ng/ml/greater, 500 pg/ml or greater, 100 pg/ml or greater, 50 pg/ml or greater, 10 pg/ml or greater, 5 pg/ml or greater, or 1 pg/ml or greater. In certain embodiments, the marking composition comprises a concentration of each of the different nucleic acids at a concentration at one of the aforementioned concentrations. In certain embodiments, the marking composition comprises a concentration of the different nucleic acids at a total concentration of one of the aforementioned concentrations.

In certain embodiments, the marking composition comprises a concentration of the plurality of isolated different nucleic acids of 2000 ng/ml or greater, 1000 ng/ml or greater, 900 ng/ml or greater, 800 ng/ml or greater, 700 ng/ml or greater, 600 ng/ml or greater, 500 ng/ml or greater, 400 ng/ml or greater, 300 ng/ml or greater, 200 ng/ml or greater, 100 ng/ml or greater, 50 ng/ml or greater, 40 ng/ml or greater, 30 ng/ml or greater, 20 ng/ml or greater, 10 ng/ml or greater, 5 ng/ml or greater, 1 ng/ml/greater, 500 pg/ml or greater, 100 pg/ml or greater, 50 pg/ml or greater, 10 pg/ml or greater, 5 pg/ml or greater, or 1 pg/ml or greater.

In certain embodiments, the marking composition comprises a concentration of one or more of the plurality of isolated different nucleic acids of 1-10 ng/ml, 1-20 ng/ml, 1-30 ng/ml, 1-40 ng/ml, 1-50 ng/ml, 1-100 ng/ml, 1-200 ng/ml, 1-300 ng/ml, 1-400 ng/ml, 1-500 ng/ml, 1-1000 ng/ml, 10-20 ng/ml, 10-30 ng/ml, 10-40 ng/ml, 10-50 ng/ml, 10-100 ng/ml, 10-200 ng/ml, 10-300 ng/ml, 10-400 ng/ml, 10-500 ng/ml, 10-1000 ng/ml, 20-30 ng/ml, 20-40 ng/ml, 20-50 ng/ml, 20-100 ng/ml, 20-200 ng/ml, 20-300 ng/ml, 20-400 ng/ml, 20-500 ng/ml, 20-1000 ng/ml, 30-40 ng/ml, 30-50 ng/ml, 30-100 ng/ml, 30-200 ng/ml, 30-300 ng/ml, 30-400 ng/ml, 30-500 ng/ml, 30-1000 ng/ml, 40-50 ng/ml, 40-100 ng/ml, 40-200 ng/ml, 40-300 ng/ml, 40-400 ng/ml, 40-500 ng/ml, 40-1000 ng/ml, 50-100 ng/ml, 50-200 ng/ml, 50-300 ng/ml, 50-400 ng/ml, 50-500 ng/ml, 50-1000 ng/ml, 100-200 ng/ml, 100-300 ng/ml, 100-400 ng/ml, 100-500 ng/ml, 100-1000 ng/ml, 200-300 ng/ml, 200-400 ng/ml, 200-500 ng/ml, 200-1000 ng/ml, 300-400 ng/ml, 300-500 ng/ml, 300-1000 ng/ml 400-500 ng/ml, 400-1000 ng/ml, or 500-1000 ng, for each different nucleic acid. In certain embodiments, the marking composition comprises a concentration of each of the different nucleic acids at a concentration at one of the aforementioned concentrations. In certain embodiments, the marking composition comprises a concentration of the different nucleic acids at a total concentration of one of the aforementioned concentrations.

In certain embodiments, the marking composition comprises a total concentration of the plurality of isolated different nucleic acids of 1-10 ng/ml, 1-20 ng/ml, 1-30 ng/ml, 1-40 ng/ml, 1-50 ng/ml, 1-100 ng/ml, 1-200 ng/ml, 1-300 ng/ml, 1-400 ng/ml, 1-500 ng/ml, 1-1000 ng/ml, 10-20 ng/ml, 10-30 ng/ml, 10-40 ng/ml, 10-50 ng/ml, 10-100 ng/ml, 10-200 ng/ml, 10-300 ng/ml, 10-400 ng/ml, 10-500 ng/ml, 10-1000 ng/ml, 20-30 ng/ml, 20-40 ng/ml, 20-50 ng/ml, 20-100 ng/ml, 20-200 ng/ml, 20-300 ng/ml, 20-400 ng/ml, 20-500 ng/ml, 20-1000 ng/ml, 30-40 ng/ml, 30-50 ng/ml, 30-100 ng/ml, 30-200 ng/ml, 30-300 ng/ml, 30-400 ng/ml, 30-500 ng/ml, 30-1000 ng/ml, 40-50 ng/ml, 40-100 ng/ml, 40-200 ng/ml, 40-300 ng/ml, 40-400 ng/ml, 40-500 ng/ml, 40-1000 ng/ml, 50-100 ng/ml, 50-200 ng/ml, 50-300 ng/ml, 50-400 ng/ml, 50-500 ng/ml, 50-1000 ng/ml, 100-200 ng/ml, 100-300 ng/ml, 100-400 ng/ml, 100-500 ng/ml, 100-1000 ng/ml, 200-300 ng/ml, 200-400 ng/ml, 200-500 ng/ml, 200-1000 ng/ml, 300-400 ng/ml, 300-500 ng/ml, 300-1000 ng/ml 400-500 ng/ml, 400-1000 ng/ml, or 500-1000 ng/ml.

Certain embodiments of the present disclosure provide a marking composition, the composition comprising a plurality of isolated different nucleic acids, the plurality of isolated different nucleic acids comprising:
a first primer region and/or a second primer region, wherein the nucleotide sequence of the first primer region and/or the nucleotide sequence of the second primer region comprise a sequence identity of 80% or less with a naturally occurring genomic nucleotide sequence; and
an intervening region between the first primer region and the second primer region;
wherein the size of the intervening region is different between the isolated different nucleic acids.

In certain embodiments, the nucleotide sequence of the first primer region and the nucleotide sequence of the second primer region are different.

In certain embodiments, the nucleotide sequence of the first primer region comprises a nucleotide sequence selected from (i) SEQ ID NO.1, the complement of SEQ ID NO.1 or a nucleotide sequence with greater than 80% sequence identity to either of the aforementioned, and/or (ii) a nucleotide sequence selected from one of SEQ ID NOs. 30 to 45 or the complement of one of the aforementioned nucleotide sequences.

In certain embodiments, the nucleotide sequence of the second primer region comprises a nucleotide sequence selected from (i) SEQ ID NO.2, the complement of SEQ ID NO.2 or a nucleotide sequence with greater than 80% sequence identity to either of the aforementioned, and/or (ii) a nucleotide sequence selected from one of SEQ ID NOs. 46 to 60 or the complement of one of the aforementioned nucleotide sequences.

In certain embodiments, the plurality of isolated different nucleic acids comprises at least two different nucleic acids

In certain embodiments, the different nucleic acids have a size of less than 500 base pairs.

In certain embodiments, the different nucleic acids have a size in the range from 100 to 300 base pairs.

In certain embodiments, the intervening region differs in size by at least 1 base pair between different nucleic acids.

In certain embodiments, the ratio of one or more of the isolated different nucleic acids to one or more other different nucleic acids is different.

In certain embodiment, the isolated different nucleic acids comprise a cleavage site for a restriction endonuclease between the first primer region and the second primer region.

In certain embodiments, the isolated different nucleic acids comprise a region of shared sequence between the first primer region and the second primer region.

In certain embodiments, the region of shared sequence comprises a cleavage site for a restriction endonuclease.

In certain embodiments, the region of shared sequence has a length of 1 to 50 base pairs.

In certain embodiments, the nucleotide sequence of the region of shared sequence comprises SEQ ID NO. 3, the complement of SEQ ID NO.3 or a nucleotide sequence with greater than 80% sequence identity to either of the aforementioned.

In certain embodiments, the ratio of one or more of the plurality of isolated different nucleic acids is different, as described herein. In certain embodiments, the ratio of one or more of the plurality of isolated different nucleic acids to one or more other different nucleic acids is the same.

Certain embodiments of the present disclosure provide a method of marking an object, the method comprising adding to the object a marking composition as described herein.

Certain embodiments of the present disclosure provide a method of marking an object with a nucleic acid tag, the method comprising adding to the object a nucleic acid as described herein.

Certain embodiments of the present disclosure provide a method of marking an object with a nucleic acid tag, the method comprising adding to an object an isolated nucleic acid comprising:
a first primer region and/or a second primer region, the first primer region and/or the second primer region comprising a sequence identity of 80% or less with a naturally occurring sequence; and
an intervening region between the first primer region and the second primer region, the intervening region having a size of at least 15 base pairs.

Certain embodiments of the present disclosure provide a method of marking an object with a nucleic acid tag, the method comprising adding to an object a plurality of isolated nucleic acids comprising:
a first primer region and/or a second primer region, the first primer region and/or the second primer region comprising a sequence identity of 80% or less with a naturally occurring genomic sequence; and
an intervening region between the first primer region and the second primer region, the intervening region having a size of at least 15 base pairs.

Isolated nucleic acids and pluralities of isolated nucleic acids are as described herein.

Methods of marking are known and include for example, applying to one or more surfaces of an object, spraying, printing, and/or painting onto an object, and incorporation into all or part of an object. In certain embodiments, the nucleic acid tag is added to an object directly. In certain embodiments, the nucleic acid tag is present as part of an ink, a glue, a paint, and/or a solution. In certain embodiments, the nucleic acid tag is present in a marking composition, as described herein.

Certain embodiments of the present disclosure provide a method of marking an object with a nucleic acid tag, the method comprising:
selecting a plurality of different nucleic acids to mark the object, the different nucleic acids having a first primer region, a second primer region and an intervening region between the first primer region and the second primer region, wherein the size of the intervening region is different between the different nucleic acids; and
adding the plurality of selected different nucleic acids to the object, thereby marking the object with a nucleic acid tag.

Selection of different nucleic acids is as described herein. In certain embodiments, the plurality of different nucleic acids comprises at least two different nucleic acids. In certain embodiments, the plurality of different nucleic acids comprises three, four, five, six, or more different nucleic acids. In certain embodiments, the plurality of different nucleic acids comprises at least three, at least four, at least five, or at least six different nucleic acids.

Sizes of the plurality of selected different nucleic acids are as described herein. In certain embodiments, one or more of the plurality of selected different nucleic acids have a size of less than 500 base pairs. In certain embodiments, the plurality of selected different nucleic acids have a size of less than 500 base pairs. In certain embodiments, one or more of the plurality of selected different nucleic acids have a size in the range from 100 to 300 base pairs. In certain embodiments, the plurality of selected different nucleic acids have a size in the range from 100 to 300 base pairs.

Primer regions and intervening regions are as described herein. In certain embodiments, the intervening region for the plurality of selected different nucleic acids differ in size by at least 5 base pairs between different nucleic acids. In certain embodiments, the intervening region for the plurality of selected different nucleic acids differ in size by at least 1 base pair between different nucleic acids. In certain embodiments, the nucleotide sequence of the first primer region and/or the nucleotide sequence of the second primer region comprise a sequence identity of 80% or less with a naturally occurring nucleotide sequence.

In certain embodiments, the nucleotide sequence of the first primer region comprises SEQ ID NO.1, the complement of SEQ ID NO.1 or a nucleotide sequence with greater than 80% sequence identity to either of the aforementioned, as described herein.

In certain embodiments, the nucleotide sequence of the second primer region comprises SEQ ID NO.2, the complement of SEQ ID NO.2 or a nucleotide sequence with greater than 80% sequence identity to either of the aforementioned, as described herein.

In certain embodiments, the amount of one or more of the plurality of selected different nucleic acids added to the object is 2000 ng or less, 1000 ng or less, 900 ng or less, 800 ng or less, 700 ng or less, 600 ng or less, 500 ng or less, 400 ng or less, 300 ng or less, 200 ng or less, 100 ng or less, 50 ng or less, 40 ng or less, 30 ng or less, 20 ng or less, 10 ng or less, 5 ng or less, 1 ng/less, 500 pg or less, 100 pg or less, 50 pg or less, 10 pg or less, 5 pg or less, or 1 pg or less. In certain embodiments, the total of the plurality of selected different nucleic acids added to the object is 2000 ng or less, 1000 ng or less, 900 ng or less, 800 ng or less, 700 ng or less, 600 ng or less, 500 ng or less, 400 ng or less, 300 ng or less, 200 ng or less, 100 ng or less, 50 ng or less, 40 ng or less, 30 ng or less, 20 ng or less, 10 ng or less, 5 ng or less, 1 ng/less, 500 pg or less, 100 pg or less, 50 pg or less, 10 pg or less, 5 pg or less, or 1 pg or less.

In certain embodiments, the amount of one or more of the plurality of selected different nucleic acids added to the object is 2000 ng or, 1000 ng or greater, 900 ng or greater, 800 ng or greater, 700 ng or greater, 600 ng or greater, 500 ng or greater, 400 ng or greater, 300 ng or greater, 200 ng or greater, 100 ng or greater, 50 ng or greater, 40 ng or greater, 30 ng or greater, 20 ng or greater, 10 ng or greater, 5 ng or greater, 1 ng/greater, 500 pg or greater, 100 pg or greater, 50 pg or greater, 10 pg or greater, 5 pg or greater, or 1 pg or greater. In certain embodiments, the total amount of the plurality of selected different nucleic acids added to the object is 2000 ng or greater, 1000 ng or greater, 900 ng or greater, 800 ng or greater, 700 ng or greater, 600 ng or greater, 500 ng or greater, 400 ng or greater, 300 ng or greater, 200 ng or greater, 100 ng or greater, 50 ng or greater, 40 ng or greater, 30 ng or greater, 20 ng or greater, 10 ng or greater, 5 ng or greater, 1 ng/greater, 500 pg or greater, 100 pg or greater, 50 pg or greater, 10 pg or greater, 5 pg or greater, or 1 pg or greater.

In certain embodiments, the amount of one or more of the plurality of selected different nucleic acids added to the object is 1-10 ng, 1-20 ng, 1-30 ng, 1-40 ng, 1-50 ng, 1-100 ng, 1-200 ng, 1-300 ng, 1-400 ng, 1-500 ng, 1-1000 ng, 10-20 ng, 10-30 ng, 10-40 ng, 10-50 ng, 10-100 ng, 10-200 ng, 10-300 ng, 10-400 ng, 10-500 ng, 10-1000 ng, 20-30 ng, 20-40 ng, 20-50 ng, 20-100 ng, 20-200 ng, 20-300 ng, 20-400 ng, 20-500 ng, 20-1000 ng, 30-40 ng, 30-50 ng, 30-100 ng, 30-200 ng, 30-300 ng, 30-400 ng, 30-500 ng, 30-1000 ng, 40-50 ng, 40-100 ng, 40-200 ng, 40-300 ng, 40-400 ng, 40-500 ng, 40-1000 ng, 50-100 ng, 50-200 ng, 50-300 ng, 50-400 ng, 50-500 ng, 50-1000 ng, 100-200 ng, 100-300 ng, 100-400 ng, 100-500 ng, 100-1000 ng, 200-300 ng, 200-400 ng, 200-500 ng, 200-1000 ng, 300-400 ng, 300-500 ng, 300-1000 ng 400-500 ng, 400-1000 ng, or 500-1000 ng, for each different nucleic acid.

In certain embodiments, the total amount of the plurality of selected different nucleic acids added to the object is 1-10 ng, 1-20 ng, 1-30 ng, 1-40 ng, 1-50 ng, 1-100 ng, 1-200 ng, 1-300 ng, 1-400 ng, 1-500 ng, 1-1000 ng, 10-20 ng, 10-30 ng, 10-40 ng, 10-50 ng, 10-100 ng, 10-200 ng, 10-300 ng, 10-400 ng, 10-500 ng, 10-1000 ng, 20-30 ng, 20-40 ng, 20-50 ng, 20-100 ng, 20-200 ng, 20-300 ng, 20-400 ng, 20-500 ng, 20-1000 ng, 30-40 ng, 30-50 ng, 30-100 ng, 30-200 ng, 30-300 ng, 30-400 ng, 30-500 ng, 30-1000 ng, 40-50 ng, 40-100 ng, 40-200 ng, 40-300 ng, 40-400 ng, 40-500 ng, 40-1000 ng, 50-100 ng, 50-200 ng, 50-300 ng, 50-400 ng, 50-500 ng, 50-1000 ng, 100-200 ng, 100-300 ng, 100-400 ng, 100-500 ng, 100-1000 ng, 200-300 ng, 200-400 ng, 200-500 ng, 200-1000 ng, 300-400 ng, 300-500 ng, 300-1000 ng 400-500 ng, 400-1000 ng, or 500-1000 ng.

In certain embodiments, the amount of one or more of the plurality of selected different nucleic acids added to the object is 10-400 ng for each different nucleic acid. In certain embodiments, the total amount of the plurality of selected different nucleic acids added to the object is 10-400 ng.

In certain embodiments, the plurality of selected different nucleic acids is present in a marking composition added to the object, as described herein.

In certain embodiments, the marking composition comprises a concentration of one or more of the plurality of selected different nucleic acids of 2000 ng/ml or less, 1000 ng/ml or less, 900 ng/ml or less, 800 ng/ml or less, 700 ng/ml or less, 600 ng/ml or less, 500 ng/ml or less, 400 ng/ml or less, 300 ng/ml or less, 200 ng/ml or less, 100 ng/ml or less, 50 ng/ml or less, 40 ng/ml or less, 30 ng/ml or less, 20 ng/ml or less, 10 ng/ml or less, 5 ng/ml or less, 1 ng/ml/less, 500 pg/ml or less, 100 pg/ml or less, 50 pg/ml or less, 10 pg/ml or less, 5 pg/ml or less, or 1 pg/ml or less. In certain embodiments, the marking composition comprises a total concentration of the plurality of selected different nucleic acids of 2000 ng/ml or less, 1000 ng/ml or less, 900 ng/ml or less, 800 ng/ml or less, 700 ng/ml or less, 600 ng/ml or less, 500 ng/ml or less, 400 ng/ml or less, 300 ng/ml or less, 200 ng/ml or less, 100 ng/ml or less, 50 ng/ml or less, 40 ng/ml or less, 30 ng/ml or less, 20 ng/ml or less, 10 ng/ml or less, 5 ng/ml or less, 1 ng/ml/less, 500 pg/ml or less, 100 pg/ml or less, 50 pg/ml or less, 10 pg/ml or less, 5 pg/ml or less, or 1 pg/ml or less.

In certain embodiments, the marking composition comprises a concentration of one or more of the plurality of selected different nucleic acids of 2000 ng/ml or greater, 1000 ng/ml or greater, 900 ng/ml or greater, 800 ng/ml or greater, 700 ng/ml or greater, 600 ng/ml or greater, 500 ng/ml or greater, 400 ng/ml or greater, 300 ng/ml or greater, 200 ng/ml or greater, 100 ng/ml or greater, 50 ng/ml or greater, 40 ng/ml or greater, 30 ng/ml or greater, 20 ng/ml or greater, 10 ng/ml or greater, 5 ng/ml or greater, 1 ng/ml/greater, 500 pg/ml or greater, 100 pg/ml or greater, 50 pg/ml or greater, 10 pg/ml or greater, 5 pg/ml or greater, or 1 pg/ml or greater. In certain embodiments, the total amount of the plurality of selected different nucleic acids added to the object is 2000 ng/ml or greater, 1000 ng/ml or greater, 900 ng/ml or greater, 800 ng/ml or greater, 700 ng/ml or greater, 600 ng/ml or greater, 500 ng/ml or greater, 400 ng/ml or greater, 300 ng/ml or greater, 200 ng/ml or greater, 100 ng/ml or greater, 50 ng/ml or greater, 40 ng/ml or greater, 30 ng/ml or greater, 20 ng/ml or greater, 10 ng/ml or greater, 5 ng/ml or greater, 1 ng/ml/greater, 500 pg/ml or greater, 100 pg/ml or greater, 50 pg/ml or greater, 10 pg/ml or greater, 5 pg/ml or greater, or 1 pg/ml or greater.

In certain embodiments, the marking composition comprises a concentration of one or more of the plurality of selected different nucleic acids of 1-10 ng/ml, 1-20 ng/ml, 1-30 ng/ml, 1-40 ng/ml, 1-50 ng/ml, 1-100 ng/ml, 1-200 ng/ml, 1-300 ng/ml, 1-400 ng/ml, 1-500 ng/ml, 1-1000 ng/ml, 10-20 ng/ml, 10-30 ng/ml, 10-40 ng/ml, 10-50 ng/ml, 10-100 ng/ml, 10-200 ng/ml, 10-300 ng/ml, 10-400 ng/ml, 10-500 ng/ml, 10-1000 ng/ml, 20-30 ng/ml, 20-40 ng/ml, 20-50 ng/ml, 20-100 ng/ml, 20-200 ng/ml, 20-300 ng/ml, 20-400 ng/ml, 20-500 ng/ml, 20-1000 ng/ml, 30-40 ng/ml, 30-50 ng/ml, 30-100 ng/ml, 30-200 ng/ml, 30-300 ng/ml, 30-400 ng/ml, 30-500 ng/ml, 30-1000 ng/ml, 40-50 ng/ml, 40-100 ng/ml, 40-200 ng/ml, 40-300 ng/ml, 40-400 ng/ml, 40-500 ng/ml, 40-1000 ng/ml, 50-100 ng/ml, 50-200 ng/ml, 50-300 ng/ml, 50-400 ng/ml, 50-500 ng/ml, 50-1000 ng/ml, 100-200 ng/ml, 100-300 ng/ml, 100-400 ng/ml, 100-500 ng/ml, 100-1000 ng/ml, 200-300 ng/ml, 200-400 ng/ml, 200-500 ng/ml, 200-1000 ng/ml, 300-400 ng/ml, 300-500 ng/ml, 300-1000 ng/ml 400-500 ng/ml, 400-1000 ng/ml, or 500-1000 ng, for each different nucleic acid.

In certain embodiments, the marking composition comprises a total concentration of the plurality of selected different nucleic acids of 1-10 ng/ml, 1-20 ng/ml, 1-30 ng/ml, 1-40 ng/ml, 1-50 ng/ml, 1-100 ng/ml, 1-200 ng/ml, 1-300 ng/ml, 1-400 ng/ml, 1-500 ng/ml, 1-1000 ng/ml, 10-20 ng/ml, 10-30 ng/ml, 10-40 ng/ml, 10-50 ng/ml, 10-100 ng/ml, 10-200 ng/ml, 10-300 ng/ml, 10-400 ng/ml, 10-500 ng/ml, 10-1000 ng/ml, 20-30 ng/ml, 20-40 ng/ml, 20-50 ng/ml, 20-100 ng/ml, 20-200 ng/ml, 20-300 ng/ml, 20-400 ng/ml, 20-500 ng/ml, 20-1000 ng/ml, 30-40 ng/ml, 30-50 ng/ml, 30-100 ng/ml, 30-200 ng/ml, 30-300 ng/ml, 30-400 ng/ml, 30-500 ng/ml, 30-1000 ng/ml, 40-50 ng/ml, 40-100 ng/ml, 40-200 ng/ml, 40-300 ng/ml, 40-400 ng/ml, 40-500 ng/ml, 40-1000 ng/ml, 50-100 ng/ml, 50-200 ng/ml, 50-300 ng/ml, 50-400 ng/ml, 50-500 ng/ml, 50-1000 ng/ml, 100-200 ng/ml, 100-300 ng/ml, 100-400 ng/ml, 100-500 ng/ml, 100-1000 ng/ml, 200-300 ng/ml, 200-400 ng/ml, 200-500 ng/ml, 200-1000 ng/ml, 300-400 ng/ml, 300-500 ng/ml, 300-1000 ng/ml 400-500 ng/ml, 400-1000 ng/ml, or 500-1000 ng/ml.

In certain embodiments, the ratio of one or more of the plurality of selected different nucleic acids to one or more other different nucleic acids is different, as described herein. In certain embodiments, the ratio of one or more of the plurality of selected different nucleic acids to one or more other different nucleic acids is the same.

In certain embodiments, the plurality of selected different nucleic acids comprises a cleavage site for a restriction endonuclease between the first primer region and the second primer region, as described herein.

In certain embodiments, the plurality of selected different nucleic acids comprise a region of shared sequence between the first primer region and the second primer region, as described herein.

In certain embodiments, the region of shared sequence comprises a cleavage site for a restriction endonuclease, as described herein.

In certain embodiments, the region of shared sequence has a length of 1 to 50 base pairs, as described herein.

In certain embodiments, the nucleotide sequence of the region of shared sequence comprises SEQ ID NO.3, the complement of SEQ ID NO.3 or a sequence with at least 80% sequence identity to the aforementioned, as described herein.

In certain embodiments, the method comprises adding a fluorescent agent to the object, as described herein.

In certain embodiments, the object is a human or animal, as described herein. In certain embodiments, the object comprises all or part of a manufactured article, as described herein.

Certain embodiments of the present disclosure provide an isolated nucleic acid comprising:
a first primer region and/or a second primer region, the first primer region and/or the second primer region comprising a sequence identity of 80% or less with a naturally occurring genomic sequence; and
an intervening region between the first primer region and the second primer region, the intervening region having a size of at least 15 base pairs.

Certain embodiments of the present disclosure provide an isolated nucleic acid comprising:
(i) SEQ ID NO.1 and/or the complement thereof; and/or
(ii) a nucleotide sequence with greater than 80% sequence identity to SEQ ID NO.1 or the complement thereof.

Certain embodiments of the present disclosure provide an isolated nucleic acid comprising:
(i) SEQ ID NO.2 and/or the complement thereof; and/or
(ii) a nucleotide sequence with greater than 80% sequence identity to SEQ ID NO.2 or the complement thereof.

Certain embodiments of the present disclosure provide an isolated nucleic acid comprising:
(i) SEQ ID NO.3 and/or the complement thereof; and/or
(ii) a nucleotide sequence with greater than 80% sequence identity to SEQ ID NO.3 or the complement thereof.

Certain embodiments of the present disclosure provide an isolated nucleic acid comprising one or more of a nucleotide sequence selected from one of SEQ ID NOs. 30 to 60, or the complement of any one of the aforementioned nucleotide sequences.

Certain embodiments of the present disclosure provide a marking composition comprising one or more of a nucleic acids as described herein.

Certain embodiments of the present disclosure provide a marking composition, the composition comprising a nucleic acid comprising:
a first primer region and/or a second primer region, the first primer region and/or the second primer region comprising a sequence identity of 80% or less with a naturally occurring genomic sequence; and
an intervening region between the first primer region and the second primer region, the intervening region having a size of at least 15 base pairs.

Certain embodiments of the present disclosure provide a marking composition, the composition comprising a nucleic acid comprising:
(i) SEQ ID NO.1 and/or the complement thereof; and/or
(ii) a nucleotide sequence with greater than 80% sequence identity to SEQ ID NO.1 and/or the complement thereof.

Certain embodiments of the present disclosure provide a marking composition, the composition comprising a nucleic acid comprising:
(i) SEQ ID NO.2 and/or the complement thereof; and/or
(ii) a nucleotide sequence with greater than 80% sequence identity to SEQ ID NO.2 and/or the complement thereof.

Certain embodiments of the present disclosure provide a marking composition, the composition comprising a nucleic acid comprising:
(i) SEQ ID NO.3 and/or the complement thereof; and/or
(ii) a nucleotide sequence with greater than 80% sequence identity to SEQ ID NO.3 and/or the complement thereof.

Certain embodiments of the present disclosure provide a marking composition, the composition comprising a nucleic acid comprising one or more of a nucleotide sequence selected from one or more of SEQ ID NOs. 30 to 60, or the complement of any one or more of the aforementioned nucleotide sequences.

Certain embodiments of the present disclosure provide a nucleic acid tag, the nucleic acid tag comprising a nucleic acid as described herein.

Certain embodiments of the present disclosure provide a method of marking an object, the method comprising tagging the object with a marking composition as described herein. Certain embodiments of the present disclosure provide an object marked with a marking composition as described herein.

Certain embodiments of the present disclosure provide an object marked with a nucleic acid tag or one or more nucleic acids as described herein.

Certain embodiments of the present disclosure provide plurality of isolated nucleic acids as described herein.

Certain embodiments of the present disclosure provide a DNA fingerprint produced by amplification of one or more nucleic acids as described herein.

Certain embodiments of the present disclosure provide a method of producing a nucleic acid for use in a tag.

Certain embodiments of the present disclosure provide a method of producing a nucleic acid for use in a tag, the method comprising:
introducing a first primer region and a second primer region as described herein into a nucleic acid; and
introducing an intervening region as described herein into the nucleic acid between the first primer region and the second primer region,

Certain embodiments of the present disclosure provide a method of producing a nucleic acid for use in a tag, the method comprising:
introducing a first primer region and a second primer region into a nucleic acid, the first primer region and/or the second primer region comprising a sequence identity of 80% or less with a naturally occurring sequence; and
introducing an intervening region into the nucleic acid between the first primer region and the second primer region, the intervening region having a size of at least 15 base pairs.

Examples of nucleic acids are as described herein. Methods for introducing nucleic acids into other nucleic acids are known, and include chemical synthesis or use of recombinant technology, for example as described in Sambrook et al. Molecular Cloning: A Laboratory Manual. 3rd ed. Cold Spring Harbor Laboratory Press, New York, N.Y. 2000 or Current Protocols in Molecular Biology. Ed. Ausubel et al. John Wiley & Sons, Inc. Cambridge, Mass., 2000.

In certain embodiments, the method of producing the nucleic acid comprises synthesis of one or more oligonucleotides. In certain embodiments, the method of producing the nucleic acid comprises synthesis of one or more oligonucleotides to all or part of one or more of the first primer region, the second primer region and the intervening region. Methods for the design and synthesis of oligonucleotides are known.

In certain embodiments, the production of the nucleic acid comprises polymerase chain reaction mediated gene synthesis. Methods for gene synthesis are known, including for example as described in Jayaraman et al. (1991) Proc. Natl. Acad. Sci. 88: 4084-4088.

In certain embodiments, the method of production of the nucleic acid comprises cloning of selected products into a compatible vector, for example a plasmid vector. Methods for isolation and cloning of nucleic acids are known and include for example Sambrook et al. Molecular Cloning: A Laboratory Manual. 3rd ed. Cold Spring Harbor Laboratory Press, New York, N.Y. 2000 or Current Protocols in Molecular Biology. Ed. Ausubel et al. John Wiley & Sons, Inc. Cambridge, Mass., 2000.

Certain embodiments of the present disclosure provide a nucleic acid produced by the method, including vectors comprising the nucleic acid and hosts comprising the vectors, as described herein.

Certain embodiments of the present disclosure provide an isolated nucleic acid comprising one or more of the following nucleotide sequences:

(i)
(SEQ ID NO. 5)
5′ CCGCTGACAAGTAAACGCGATTGATCACATTTTGGTGAGAAGTTGTCCAGAC
TTAAGGACAGTGATTCAAGACGTTCTAGTCGGTCCCAAACCTGACATGCATGGGC
GCGCAACCTCTAAACCTTAACCTCAGAAGTGAGTGCTTCCTGGAACCTCTAGTCT
GGATCCCGGCAGCTACCTCGGACGGTTGAGAGGAGTACCCTAGTTCGCTGCCAG
GCGTATATCCCGCAATCAACATCCCTGTATAAATTAGTTGTGGATATTTCAACAT
ATTGCTATGGTCCGACTTTTCAGTAGGCA 3′;
(ii)
(SEQ ID NO. 6)
5′ CCGCTGACAAGTAAACGCGATTGATCACATTTTGGTGAGA 3′;
(iii)
(SEQ ID NO. 7)
5′ AGTTGTCCAGACTTAAGGAC 3′;
(iv)
(SEQ ID NO. 8)
5′ AGTTGTCCAGACTTAAGGACAGTGA 3′;
(v)
(SEQ ID NO. 9)
5′ AGTTGTCCAGACTTAAGGACAGTGATTCAA 3′;
(vi)
(SEQ ID NO. 10)
5′ ACATGCATGGGCGCGCAACCTCTAAACCTTAACCTCAGAAGTGAG 3′;
(vii)
(SEQ ID NO. 11)
5′ ACATGCATGGGCGCGCAACCTCTAAACCTTAACCTCAGAAGTGAGTGCTT 3′;
(SEQ ID NO. 12)
5′ AGTTGTCCAGACTTAAGGACAGTGATTCAAGACGTTCTAGTCGGTCCCAAACCTG 3′;
(viii)
(SEQ ID NO. 13)
5′ ACATGCATGGGCGCGCAACCTCTAAACCTTAACCTCAGAAGTGAGTGCTTCCTGG 3′;
(ix)
(SEQ ID NO. 14)
5′ AACCTCTAGTCTGGATCCCGGCAGCTACCTCGGACGGTTGAGAGGAGTACCCTAG 3′;
(x)
(SEQ ID NO. 15)
5′ TTCGCTGCCAGGCGTATATCCCGCAATCAACATCCCTGTATAAATTAGTTGTGGA 3′;
(xi)
(SEQ ID NO. 16)
5′ TATTTCAACATATTGCTATGGTCCGACTTTTCAGTAGGCA 3′;
(xii)
(SEQ ID NO. 17)
5′ ATCAATCGCGTTTACTTGTCAGCGG 3′;
(xiii)
(SEQ ID NO. 18)
5′ CAATATGTTGAAATAGTCCTTAAGTCTGGACAACTTCTCACCAAAATGTG 3′;
(xiv)
(SEQ ID NO. 19)
5′ CAATATGTTGAAATATCACTGTCCTTAAGTCTGGACAACTTCTCACCAAAATGTG 3′;
(xv)
(SEQ ID NO. 20)
5′CAATATGTTGAAATATTGAATCACTGTCCTTAAGTCTGGACAACTTCTCACCAAAATGTG 3′;
(xvi)
(SEQ ID NO. 21)
5′ CAATATGTTGAAATACCAGGAAGCACTCACTTCTGAGGTT 3′;
(xvii)
(SEQ ID NO. 22)
5′ TAGCTGCCGGGATCCAGACTAGAGGTTCTCACTTCTGAGGTT 3′;
(xviii)
(SEQ ID NO. 23)
5′-TAGCTGCCGGGATCCAGACTAGAGGTTAAGCACTCACTTCTGAGGTT-3′;
(xix)
(SEQ ID NO. 24)
5′ CTTGAATCACTGTCCTTAAGTCTGGACAACTTCTCACCAAAATGTG 3′;
(xx)
(SEQ ID NO. 25)
5′ AAGGTTTAGAGGTTGCGCGCCCATGCATGTCAGGTTTGGGACCGACTAGAACGT 3′;
(xxi)
(SEQ ID NO. 26)
5′ TAGCTGCCGGGATCCAGACTAGAGGTTCCAGGAAGCACTCACTTCTGAGGTT 3′;
(xxi)
(SEQ ID NO. 27)
5′-GCGGGATATACGCCTGGCAGCGAACTAGGGTACTCCTCTCAACCGTCCGAGG-3′;
(xxiii)
(SEQ ID NO. 28)
5′ CAATATGTTGAAATATCCACAACTAATTTATACAGGGATGTTGATT 3′;
(xxiv)
(SEQ ID NO. 29)
5′ TGCCTACTGAAAAGTCGGACCATAG 3′;

and/or a nucleotide sequence with greater than 80% sequence identity to any of the aforementioned nucleotide sequences or the complement thereof. Other levels of sequence identity are described herein.

### Example 1

### Design of Template and Primers

A random sequence generator was used to design the sequence of the longest 250 bp inter-primer region (otherwise referred to as an ‘intervening region’). The DNA sequence of this inter-primer region generated by this process was as follows: 5′-CACATTTTGGTGAGAAGTTGTCCAGACTTAAGGACAGTGATTCAAGACGTTCTAG TCGGTCCCAAACCTGACATGCATGGGCGCGCAACCTCTAAACCTTAACCTCAGAA GTGAGTGCTTCCTGGAACCTCTAGTCTGGATCCCGGCAGCTACCTCGGACGGTTG AGAGGAGTACCCTAGTTCGCTGCCAGGCGTATATCCCGCAATCAACATCCCTGTA TAAATTAGTTGTGGATATTTCAACATATTG-3′ (SEQ ID NO.3).

A 35 bp region of this 250 bp region was selected to be a ‘fixed’ region (otherwise referred to as a ‘region of shared sequence’). This contained a AflII site unique to the 250 bp sequence. The DNA sequence of the fixed region was as follows:

Af1II
(SEQ ID NO. 4)
5′-CACATTTTGGTGAGAAGTTGTCCAGACTTAAGGAC-3′.

A random sequence generator was also used to design the sequence of 25 bp forward and reverse flanking sequence to be included in each molecule.

150 candidate primer sequences selected on basis of a GC content of 48%, and Tm of 74° C. according to the formula Tm=81.5+(0.41x % GC×100)−675/25.

Each of these primer sequences was then analysed by nucleic acid-nucleic acid BLASTN (http://www.ncbi.nlm.nih.gov/blast/Blast.cgi?PAGE=Nucleotides) against the Nucleotide collection (nr/nt) and Human genomic plus transcript (Human G+T) databases. 5 sequences giving alignment scores <40 against nr/nt, <26 against Human G+T, and no homology in the 3′-most 2 bases were selected. These 5 sequences were then checked for propensity to form intra-molecular interactions (hairpin, self-dimer), and inter-molecular interactions (primer dimer) in pair-wise fashion. They were also checked for lack of homology to the 250 bp inter-primer sequence and pUC plasmid backbone. The two primers selected showed the lowest degree of undesirable interactions.

The DNA sequences of the primers were as follows:

Primer 1:
(SEQ ID NO. 1)
5′-CCGCTGACAAGTAAACGCGATTGAT-3′
Primer 2:
(SEQ ID NO. 2)
5′-TGCCTACTGAAAAGTCGGACCATAG-3′

Together the inter primer region and the two primer sequences formed a DNA molecule of 300 bp, with a DNA sequence as follows:

(SEQ ID NO. 5)
5′ CCGCTGACAAGTAAACGCGATTGATCACATTTTGGTGAGAAGTT
GTCCAGACTTAAGGACAGTGATTCAAGACGTTCTAGTCGGTCCCAAA
CCTGACATGCATGGGCGCGCAACCTCTAAACCTTAACCTCAGAAGTG
AGTGCTTCCTGGAACCTCTAGTCTGGATCCCGGCAGCTACCTCGGAC
GGTTGAGAGGAGTACCCTAGTTCGCTGCCAGGCGTATATCCCGCAAT
CAACATCCCTGTATAAATTAGTTGTGGATATTTCAACATATTGCTAT
GGTCCGACTTTTCAGTAGGCA-3′

Following design of the 250 bp (300 bp including flanking Primers 1 and 2) template, shorter templates were designed by omitting sequence from one or more areas of the variable region. This involved omission of, or re-design and synthesis, of one or more oligos.

### Example 2

### Generation of Tagging DNA Molecules

DNA molecules were produced essentially as described in Jarayaman et al. PNAS, 88, 4084-4088 (1991). This procedure denatures, anneals then ligates oligos together in the correct order. The desired sequence is then amplified up using flanking primers for subsequent cloning and sequence verification.

Sequence of oligos were selected by ‘breaking up’ top and bottom strands of each molecule into 20-60b long sequences. Overlapping bottom strand oligos were used to provide a template on which top strand oligos can correctly anneal. Top strand oligos were then ligated together, the mix diluted, then flanking primers TL uni and BR uni used to amplify the ligated sequence using a proof reading high fidelity polymerase.

The oligonucleotides utilised in this work for each of the constructs is provided in Table 1.

TABLE 1
Oligos used in each gene construction

| 100 bp | 105 bp | 110 bp | 190 bp | 290 bp | 295 bp | 300 bp |
|---|---|---|---|---|---|---|
| TL uni | TL uni | TL uni | TL uni | TL uni | TL uni | TL uni |
| T100A | T105 | T110 | T300A | T300A | T300A | T300A |
| TR uni | TR uni | TR uni | T300B | T290 | T295 | T300B |
| BL uni | BL uni | BL uni | TR uni | T300C | T300C | T300C |
| B100A | B105 | B110 | BL uni | T300D | T300D | T300D |
| BR uni | BR uni | BR uni | B300A | TR uni | TR uni | TR uni |
| | | | B300B | BL uni | BL uni | BL uni |
| | | | B190 | B300A | B300A | B300A |
| | | | BR uni | B300B | B300B | B300B |
| | | | | B290 | B295 | B300C |
| | | | | B300D | B300D | B300D |
| | | | | B300E | B300E | B300E |
| | | | | BR uni | BR uni | BR uni |

The nucleotide sequences of the various oligonucleotides used are shown in Table 2.

TABLE 2
Oligo sequences (5′ to 3′)

| | |
|---|---|
| TL uni | CCGCTGACAAGTAAACGCGATTGATCACATTTTGGTGAGA (SEQ ID NO.6) |
| T100A | 5′Phosphate-AGTTGTCCAGACTTAAGGAC (SEQ ID NO.7) |
| T105 | 5′Phosphate-AGTTGTCCAGACTTAAGGACAGTGA (SEQ ID NO. 8) |
| T110 | 5′Phosphate-AGTTGTCCAGACTTAAGGACAGTGATTCAA (SEQ ID NO. 9) |
| T290 | 5′Phosphate-ACATGCATGGGCGCGCAACCTCTAAACCTTAACCTCAGAAGTGAG |
| | (SEQ ID NO. 10) |
| T295 | 5′Phosphate- |
| | ACATGCATGGGCGCGCAACCTCTAAACCTTAACCTCAGAAGTGAGTGCTT |
| | (SEQ ID NO. 11) |
| T300A | 5′Phosphate- |
| | AGTTGTCCAGACTTAAGGACAGTGATTCAAGACGTTCTAGTCGGTCCCAAACCTG |
| | (SEQ ID NO. 12) |
| T300B | 5′Phosphate- |
| | ACATGCATGGGCGCGCAACCTCTAAACCTTAACCTCAGAAGTGAGTGCTTCCTGG |
| | (SEQ ID NO. 13) |
| T300C | 5′Phosphate- |
| | AACCTCTAGTCTGGATCCCGGCAGCTACCTCGGACGGTTGAGAGGAGTACCCTAG |
| | (SEQ ID NO. 14) |
| T300D | 5′Phosphate- |
| | TTCGCTGCCAGGCGTATATCCCGCAATCAACATCCCTGTATAAATTAGTTGTGGA |
| | (SEQ ID NO. 15) |
| TR uni | 5′Phosphate-TATTTCAACATATTGCTATGGTCCGACTTTTCAGTAGGCA |
| | (SEQ ID NO. 16) |
| BL uni | ATCAATCGCGTTTACTTGTCAGCGG (SEQ ID NO. 17) |
| B100A | CAATATGTTGAAATAGTCCTTAAGTCTGGACAACTTCTCACCAAAATGTG |
| | (SEQ ID NO. 18) |
| B105 | CAATATGTTGAAATATCACTGTCCTTAAGTCTGGACAACTTCTCACCAAAATGTG |
| | (SEQ ID NO. 19) |
| B110 | CAATATGTTGAAATATTGAATCACTGTCCTTAAGTCTGGACAACTTCTCACCAAAATGTG |
| | (SEQ ID NO. 20) |
| B190 | CAATATGTTGAAATACCAGGAAGCACTCACTTCTGAGGTT (SEQ ID NO. 21) |
| B290 | TAGCTGCCGGGATCCAGACTAGAGGTTCTCACTTCTGAGGTT (SEQ ID NO. 22) |
| B295 | TAGCTGCCGGGATCCAGACTAGAGGTTAAGCACTCACTTCTGAGGTT |
| | (SEQ ID NO. 23) |
| B300A | CTTGAATCACTGTCCTTAAGTCTGGACAACTTCTCACCAAAATGTG |
| | (SEQ ID NO. 24) |
| B300B | AAGGTTTAGAGGTTGCGCGCCCATGCATGTCAGGTTTGGGACCGACTAGAACGT |
| | (SEQ ID NO. 25) |
| B300C | TAGCTGCCGGGATCCAGACTAGAGGTTCCAGGAAGCACTCACTTCTGAGGTT |
| | (SEQ ID NO. 26) |
| B300D | GCGGGATATACGCCTGGCAGCGAACTAGGGTACTCCTCTCAACCGTCCGAGG |
| | (SEQ ID NO. 27) |
| B300E | CAATATGTTGAAATATCCACAACTAATTTATACAGGGATGTTGATT |
| | (SEQ ID NO. 28) |
| BR uni | TGCCTACTGAAAAGTCGGACCATAG (SEQ ID NO. 29) |

Following amplification, the correct size amplicons were extracted by agarose gel electrophoresis and cloned into pAcquire (Alchemy Biosciences) using the recommended blunt PCR product cloning protocol.

2-4 clones were propagated for each molecule, plasmid DNA extracted and sequence checked in both directions using primers directed at insert-flanking SP6 and T7 sites in the vector.

One correct sequence clone was selected for large scale plasmid DNA preparation.

In the Examples described herein, reference to a DNA tag/template/molecule refers to a plasmid (pAcquire) molecule containing the cloned DNA insert between 100 bp and 300 bp in length (including Primer 1 and Primer 2 sequences).

Large scale plasmid preparations were tested by sequence verification using SP6 and T7 primers, and amplification of the cloned insert using Primer 1 and Primer 2. This was done using AmpFISTR Profiler Plus Master Mix (Applied Biosystems) using their recommended protocol but with substituted primers and 35 cycle reaction.

PCR amplifications performed using Primer 1 and Primer 2 referred to herein were conducted using 5′ 6-FAM labelled Primer 1. This allowed detection of amplicon DNA molecules without use of intercalating stains such as ethidium bromide, including detection by denaturing capillary electrophoresis, which is the method generally used by forensic authorities. For convenience and more sensitive detection, however, most of the data presented results from ethidium bromide staining of non-denatured DNA separated on agarose of polyacrylamide gels.

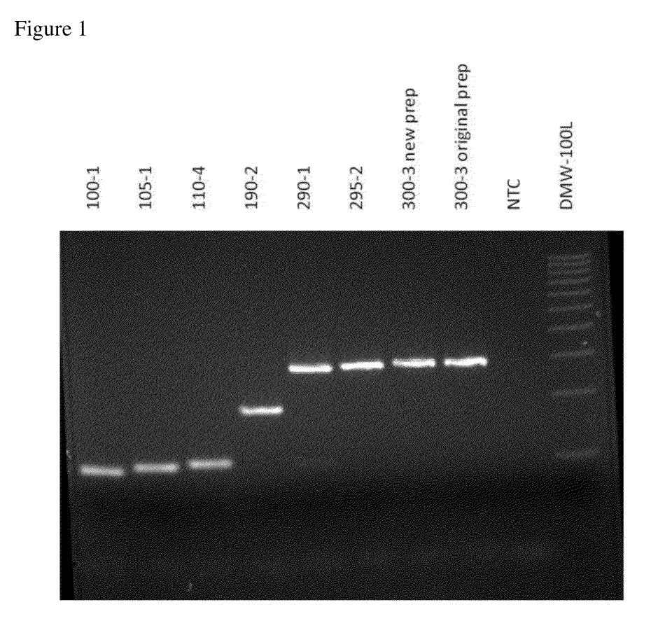

FIG. 1 shows the amplification products from individual plasmid templates, confirming that separately they can be amplified correctly and produce products of the correct size. Templates 100, 105, 110, 190, 290, 295 and 300 (2 different preparations) generated the expected 100 bp, 105 bp, 110 bp, 190 bp, 290 bp, 295 bp and 300 bp products respectively. A DNA marker containing 100-1000 bp DNA in 100 bp increments and was used to confirm the size of the different PCR products.

### Example 3

### Method for Detection of Templates by PCR Using AmpFISTR Profiler Plus Master Mix

One correct sequence clone was selected for each construct and large scale plasmid DNA preparation performed. The reaction conditions for detection of templates using AmpFISTR Profiler Plus Master Mix were as shown in Table 3

TABLE 3

| | ul per | | ul per |
|---|---|---|---|
| 50 ul reaction volume | reaction | 25 ul reaction volume | reaction |
| AmpFlSTR Profiler | 21 | AmpFlSTR Profiler | 10.5 |
| Plus Mastermix | | Plus Mastermix | |
| AmpliTaq Gold | 1 | AmpliTaq Gold | 0.5 |
| 10 uM Primer 1 | 3 | 10 uM Primer 1 | 1.5 |
| (5′ FAM labelled)* | | (5′ FAM labelled)* | |
| 10 uM Primer 2 | 3 | 10 uM Primer 2 | 1.5 |
| Water | 5 | Water | 2.5 |
| Mix above, add 30 | | Mix above, add 15 | |
| ul to 20 ul DNA | | ul to 10 ul DNA | |

35 cycle PCR reaction

| Step # | |
|---|---|
| 1 | 95° C. 11 min |
| 2 | 94° C. 1 min |
| 3 | 59° C. 1 min |
| 4 | 72° C. 1 min |
| 5 | Go to 2 34 more times |
| 6 | 60° C. 45 min |
| 7 | 25° C. forever |

Run aliquot on directly gels or analyse by capillary electrophoresis

### Example 4

### Generation of Mixtures of Tag DNA Molecules

Initial testing of PCR conditions, detection sensitivity, detection specificity and template stability in different buffer formulations involved 100 bp and 300 bp insert templates only, either individually or in combination. A default concentration of 200 ng/ul of each molecule was chosen arbitrarily for these tests.

When mixed together, detection sensitivity was generally higher for shorter templates as judged by the number of amplicon molecules produced in the reaction. This lead to testing the effect of adding relatively more of the longer templates to mixes, with a view to balancing the amount of different sized templates.

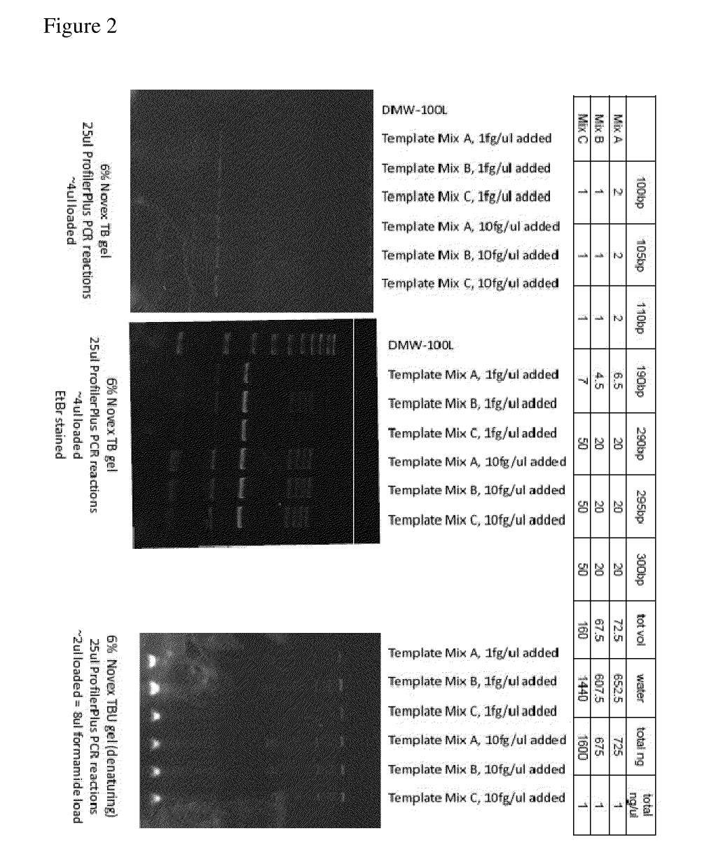

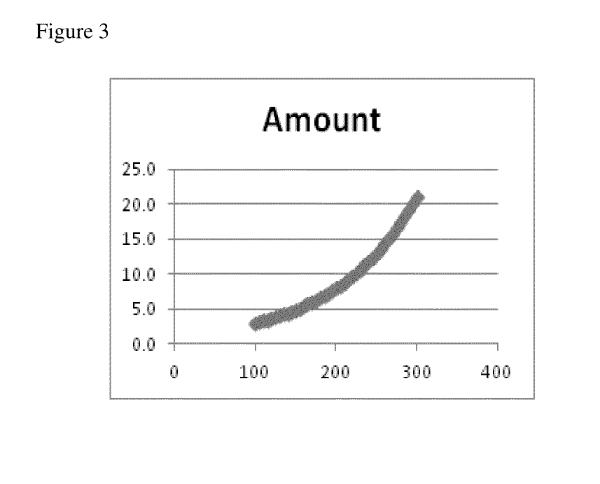

FIG. 2 shows one experiment using different amounts of 100 bp, 105 bp, 110 bp, 190 bp, 290 bp, 295 bp and 300 bp templates mixed together. Following PCR, the intensity of fluorescence from the 6-FAM Primer 1 incorporated into each molecule allows a comparative analysis of the approximate amount of each product generated. By varying the relative amount of each template in a mixture it was possible to estimate how much extra of the longer templates was required to ‘balance’ the PCR reactions, such that the same number of molecules of each product result. By comparison of the results from template mixtures with the same ratio of template quantity, but different total amounts (10 fg or 100 fg added), this experiment also showed that this balance was not significantly affected by the total amount of DNA in the reaction. On the basis of this data, the amount of each template used in a tag may be selected on the basis as determined by reference to FIG. 3.

### Example 5

### Specificity of Detection

The specificity of PCR to the designed template was tested by inclusion of potentially sample-contaminating DNA in PCR reactions.

50 ul 35 cycle PCR reactions using AmpFISTR ProfilerPlus PCR mix and AmpliTaq Gold polymerase were performed containing 20 fg and 400 fg of 100 bp and 300 bp template, in the presence or absence of 1 ng human, grass or soil genomic DNA.

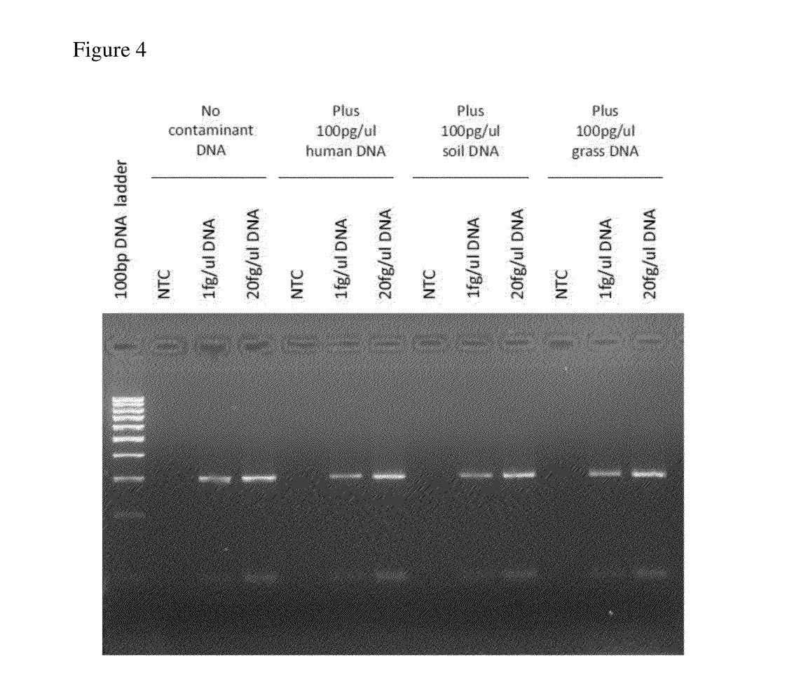

The results are shown in FIG. 4, which shows the results of AmpFISTR PCR reactions with and without 100 and 300 plasmid template in the presence or absence of 1 ng of genomic human, plant (lawn grass) and soil DNA. The presence of contaminating DNA does not result in PCR products with or without specific templates present, and the amplification products were not changed by addition of the contaminant DNA.

### Example 6

### Sensitivity of Detection

The sensitivity of detection of template was tested by amplification of different amounts of template, and varying the number of PCR cycles.

50 ul 28 and 35 cycle PCR reactions using AmpFISTR Profiler Plus PCR mix and AmpliTaq Gold polymerase were performed containing 20 fg, 100 fg, 400 fg and 2000 fg of 100 bp and 300 bp template.

Detection sensitivity with 35 cycles was approximately 100× higher than with 28 cycles of PCR amplification. A large amount of both amplicons was produced from 20 fg template in the 35 cycle reaction. A similar amount of both amplicons was generated from 2000 fg template in the 28 cycle reaction, indicating that the 35 cycle reaction is approximately 100 times more sensitive.

### Example 7

### Detection of Template DNA by Swab Sampling and PCR from Various Surfaces at Different Times after Spraying

Detection sensitivity with 100 ug of 100 bp and 300 bp DNA templates were diluted to 500 ml in the following buffer mixture: 1 mM Tris pH 8.0, and 0.1 mM EDTA, pH 8.0.

This mixture was loaded into a spray device and sprayed onto various surfaces from a distance of approximately 1 metre, so as to mimic exposure to a triggered spray device. Cotton buds soaked in 200 ul of 10 mM Tris, pH8.0, 1 mM EDTA were used at various times after spraying to swab DNA from the sprayed surfaces.

After swabbing, cotton buds were returned to the tube containing TE buffer.

Sampling from swabs was performed by removal of 20 ul of buffer surrounding the swab. Detection was done by addition of the sample to 50 ul 35 cycle PCR reactions using AmpFISTR ProfilerPlus reagent and AmpliTaq Gold.

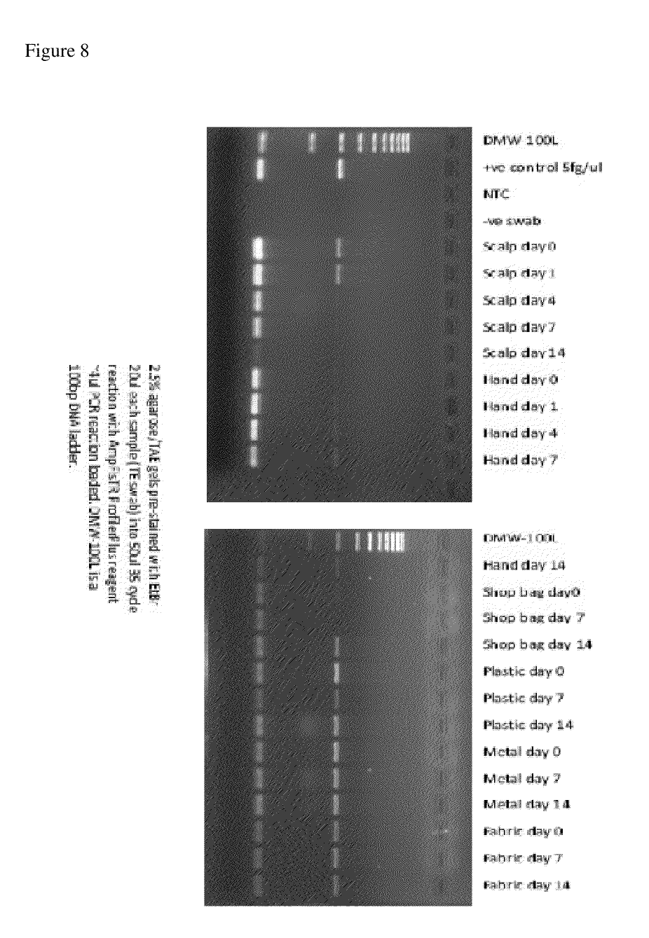

Detection of template was possible on inanimate objects for at least 14 days with minimal reduction in amount of amplicon generated from both 100 bp and 300 bp templates. Detection of template was also possible from human scalp and back of hand, despite daily washing, for at least 14 days. The results are shown in FIG. 8. 4 ul of each PCR reaction was loaded on a 2.5% agarose/TAE gels pre-stained with ethidium bromide. A negative control (buffer from an unused swab) gave no products. A positive control containing 100 fg of each template was included to check reaction performance. Products of the expected size (100 bp and 300 bp) are detected by this method in all samples over the course of this study, though the amount generally declines with the time from exposure to spray on Day 0. Swab samples taken from human skin showed biased detection of the 100 bp template. There is some variability in the result between different sampling from the same surface, which is consistent with the expected non-uniform coverage by the sprayed DNA.

### Example 8

### Analysis by Capillary Electrophoresis

Detection of amplicon DNA utilised a 5′ FAM label on Primer 1. AmpFISTR Profiler Plus PCR reactions were performed on the combinations of template shown in Table 4.

TABLE 4

| Sample ref # | Templates included (200 fg each/reaction) |
|---|---|
| A | 100bp, 105bp |
| B | 200bp. 110bp |
| C | 100bp, 105bp, 110bp, 190bp, 290bp, 295bp, 300bp |
| D | 100bp, 110bp, 190bp, 290bp, 300bp |
| E | 100bp, 190bp, 300bp |
| F | 100bp, 110bp, 290bp, 300bp |
| 1 | 100bp |
| 2 | 105bp |
| 3 | 110bp |
| 4 | 190bp |
| 5 | 290bp |
| 6 | 295bp |
| 7 | 300bp (template preparation #2) |
| 8 | 300bp (template preparation #1) |

Unpurified PCR reactions were supplied to 1st BASE Pte Ltd, Singapore, for analysis on Applied Biosystems 3730xl capillary DNA sequencing instrumentation or similar as part of their commercially available Fragment Analysis service (see http://www.base-asia.com/fragment_analysis/).

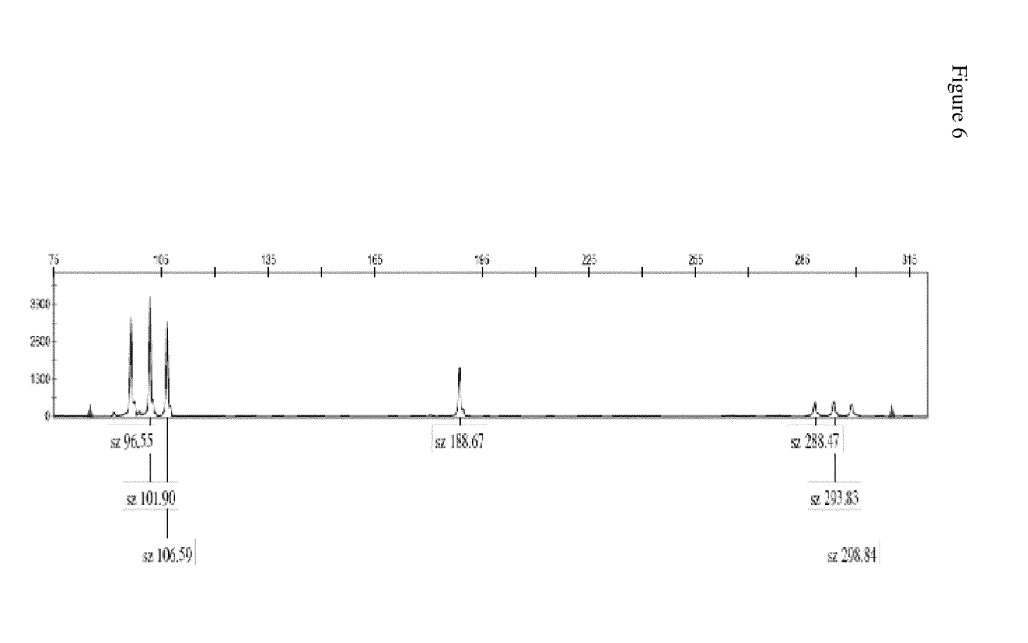

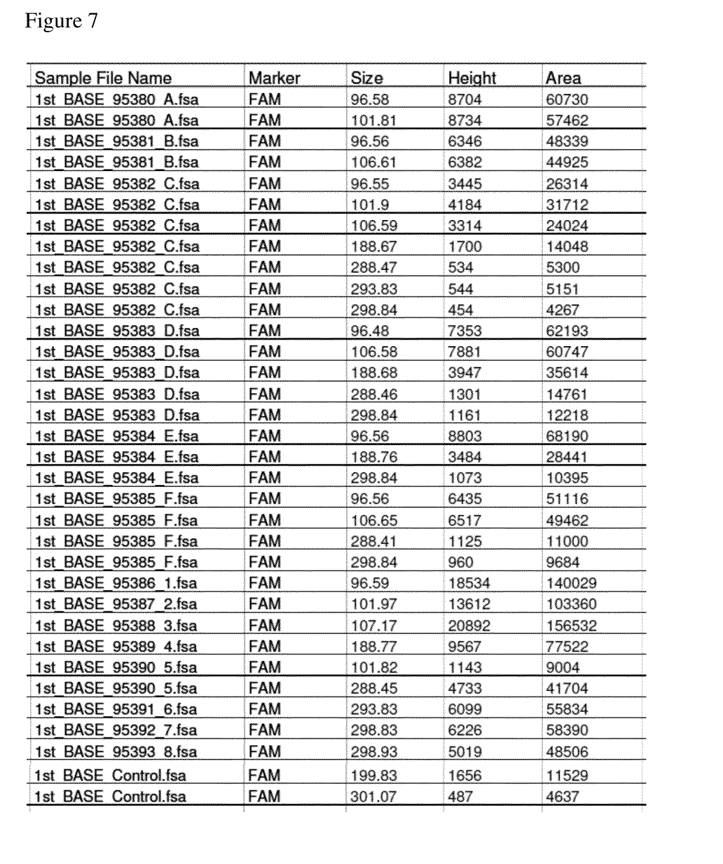

A representative trace for sample reference #6 is shown in FIG. 6. Each peak on the trace corresponds to an individual PCR product, and estimated sizes correspond to expected sizes +/−<2 bp. The peaks detected for each of the samples is shown in FIG. 7. Each peak detected and reported corresponds to an individual PCR product, and estimated sizes correspond to expected sizes +/−<2.2 bp.

### Example 9

### Marking Composition

An example of a marking composition is as follows:
(i) 1 ng/ml to 40 ug/ml of each separate DNA species (one or more), with typically the concentration of each separate DNA species being in the range from 1 ng/ml to 1 ug/ml;
(ii) 1 mM to 50 mM Tris pH8.0, typically 10 mM Tris pH 8.0;
(iii) 0.1 mM to 10 mM EDTA, typically 1 mM EDTA; and
(iv) optionally 0.01% to 1% Tween 20, typically 0.1% Tween 80.

Reference to any prior art in this specification is not, and should not be taken as, an acknowledgment or any form of suggestion that this prior art forms part of the common general knowledge in any country.

Also, it must be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1-97. (canceled)

98. A method of identifying an object marked with a nucleic acid tag, the nucleic acid tag comprising a plurality of selected different nucleic acids, the method comprising:
amplifying nucleic acids in the nucleic acid tag, wherein the amplification of the plurality of selected different nucleic acids in the nucleic acid tag produces a plurality of amplification products with a different size;
detecting the plurality of selected different nucleic acids in the nucleic acid tag by the size of the plurality of amplification products; and
identifying the object marked with the nucleic acid tag by the presence of the plurality of selected different nucleic acids in the nucleic tag.

99. The method according to claim 98, wherein the plurality of selected different nucleic acids comprise a first primer region and/or a second primer region for binding of a primer for amplification of nucleic acids in the nucleic acid tag.

100. The method according to claim 99, wherein the nucleotide sequence of the first primer region and/or the nucleotide sequence of the second primer region comprising a sequence identity of 80% or less with a naturally occurring genomic nucleotide sequence.

101. The method according to claim 99, wherein the nucleotide sequence of the first primer region comprises a nucleotide sequence selected from (i) SEQ ID NO.1, the complement of SEQ ID NO.1 or a nucleotide sequence with greater than 80% sequence identity to either of the aforementioned, and/or (ii) a nucleotide sequence selected from one of SEQ ID NOs. 30 to 45 or the complement of one of the aforementioned nucleotide sequences.

102. The method according to claim 99, wherein the nucleotide sequence of the second primer region comprises a nucleotide sequence selected from (i) SEQ ID NO.2, the complement of SEQ ID NO.2 or a nucleotide sequence with greater than 80% sequence identity to either of the aforementioned, and/or (ii) a nucleotide sequence selected from one of SEQ ID NOs. 46 to 60 or the complement of one of the aforementioned nucleotide sequences.

103. The method according to claim 99, wherein the selected different nucleic acids comprise an intervening region between the first primer region and the second primer region, the size of the intervening region being different between the selected different nucleic acids.

104. The method according to claim 103, wherein the intervening region has a size in the range from 50 to 500 base pairs.

105. The method according to claim 99, wherein the selected different nucleic acids comprises a cleavage site for a restriction endonuclease between the first primer region and the second primer region and the method further comprises cleaving the amplification products with the restriction endonuclease.

106. The method according to claim 98, wherein the nucleic acid tag comprises plasmid vectors comprising the selected different nucleic acid acids.

107. A method of determining whether an object has been marked with a nucleic acid tag, the nucleic acid tag comprising a plurality of selected different nucleic acids, the method comprising:
obtaining a sample from the object;
amplifying nucleic acids in the sample, wherein amplification of the plurality of selected different nucleic acids in the nucleic acid tag produces a plurality of amplification products with a different size;
detecting the plurality of selected different nucleic acids in the nucleic acid tag by the size of the plurality of amplification products; and
determining whether the object has been marked with the nucleic acid tag by the presence of the plurality of selected different nucleic acids in the nucleic tag.

108. A plurality of isolated different nucleic acids, the plurality of different nucleic acids comprising:
a first primer region and a second primer region, wherein the nucleotide sequence of the first primer region and/or the nucleotide sequence of the second primer region comprise a sequence identity of 80% or less with a naturally occurring genomic nucleotide sequence; and
an intervening region between the first primer region and the second primer region; wherein the size of the intervening region is different between the different nucleic acids.

109. The plurality of isolated different nucleic acids according to claim 108, wherein the nucleotide sequence of the first primer region comprises a nucleotide sequence selected from (i) SEQ ID NO.1, the complement of SEQ ID NO.1 or a nucleotide sequence with greater than 80% sequence identity to either of the aforementioned, and/or (ii) a nucleotide sequence selected from one of SEQ ID NOs. 30 to 45 or the complement of one of the aforementioned nucleotide sequences.

110. The plurality of isolated different nucleic acids according to claim 108, wherein the nucleotide sequence of the second primer region comprises a nucleotide sequence selected from (i) SEQ ID NO.2, the complement of SEQ ID NO.2 or a nucleotide sequence with greater than 80% sequence identity to either of the aforementioned, and/or (ii) a nucleotide sequence selected from one of SEQ ID NOs. 46 to 60 or the complement of one of the aforementioned nucleotide sequences.

111. The plurality of isolated different nucleic acids according to claim 108, wherein the intervening region has a size in the range from 50 to 500 base pairs.

112. The plurality of isolated different nucleic acids according to claim 108, wherein the different nucleic acids comprises a cleavage site for a restriction endonuclease between the first primer region and the second primer region.

113. The plurality of isolated different nucleic acids according to claim 108, wherein the different nucleic acids comprise plasmid vectors comprising the different nucleic acid acids.

114. A marking composition comprising a plurality of isolated different nucleic acids according to claim 108.

115. A nucleic acid tag for marking an object, the nucleic acid tag comprising a plurality of isolated different nucleic acids according to claim 108.

116. A method of marking an object with a nucleic acid tag, the method comprising adding a plurality of isolated different nucleic acids according to claim 108 to the object.

117. A DNA fingerprint produced by amplification of the plurality of isolated different nucleic acids according to claim 108.


SEQUENCE LISTING (60 sequences)

SEQ ID NO: 1 - DNA, 25 bp, Artificial Sequence
  Primer Sequence
ccgctgacaagtaaacgcgattgat

SEQ ID NO: 2 - DNA, 25 bp, Artificial Sequence
  Primer Sequence
tgcctactgaaaagtcggaccatag

SEQ ID NO: 3 - DNA, 248 bp, Artificial Sequence
  Intervening Region
cacattttggtgagaagttgtccagacttaaggacagtgattcaagacgttctagtcggtcccaaacctgacatgcatgggcgcgcaacctctaaaccttaactcagaagtgagtgcttcctggaacctctagtctggatcccggcagctacctcggacggttgagaggagtaccctagttcgctgccaggcgtatatcccgcaatcaacatccctgtataaattagtttggatatttcaacatattg

SEQ ID NO: 4 - DNA, 35 bp, Artificial Sequence
  Region of shared sequence
cacattttggtgagaagttgtccagacttaaggac

SEQ ID NO: 5 - DNA, 300 bp, Artificial Sequence
  300 bp construct
ccgctgacaagtaaacgcgattgatcacattttggtgagaagttgtccagacttaaggacagtgattcaagacgttctagtcggtcccaaacctgacatgcatgggcgcgcaacctctaaaccttaacctcagaagtgagtgcttcctggaacctctagtctggatcccggcagctacctcggacggttgagaggagtaccctagttcgctgccaggcgtatatcccgcaatcaacatccctgtataaattagttgtggatatttcaacatattgctatggtccgacttttcagtaggca

SEQ ID NO: 6 - DNA, 40 bp, Artificial Sequence
  TL uni oligonucleotide sequence
ccgctgacaagtaaacgcgattgatcacattttggtgaga

SEQ ID NO: 7 - DNA, 20 bp, Artificial Sequence
  T100A oligonucleotide sequence
agttgtccagacttaaggac

SEQ ID NO: 8 - DNA, 25 bp, Artificial Sequence
  T105 oligonulceotide sequence
agttgtccagacttaaggacagtga

SEQ ID NO: 9 - DNA, 30 bp, Artificial Sequence
  T110 oligomucleotide sequence
agttgtccagacttaaggacagtgattcaa

SEQ ID NO: 10 - DNA, 45 bp, Artificial Sequence
  T290 oligonucleotide sequence
acatgcatgggcgcgcaacctctaaaccttaacctcagaagtgag

SEQ ID NO: 11 - DNA, 50 bp, Artificial Sequence
  T295 oligonucleotide sequence
acatgcatgggcgcgcaacctctaaaccttaacctcagaagtgagtgctt

SEQ ID NO: 12 - DNA, 55 bp, Artificial Sequence
  T300A oligonucleotide sequence
agttgtccagacttaaggacagtgattcaagacgttctagtcggtcccaaacctg

SEQ ID NO: 13 - DNA, 55 bp, Artificial Sequence
  T300B oligonucleotide sequence
acatgcatgggcgcgcaacctctaaaccttaacctcagaagtgagtgcttcctgg

SEQ ID NO: 14 - DNA, 55 bp, Artificial Sequence
  T300C oligonucleotide sequence
aacctctagtctggatcccggcagctacctcggacggttgagaggagtaccctag

SEQ ID NO: 15 - DNA, 55 bp, Artificial Sequence
  T300D oligonucleotide sequence
ttcgctgccaggcgtatatcccgcaatcaacatccctgtataaattagttgtgga

SEQ ID NO: 16 - DNA, 40 bp, Artificial Sequence
  TR uni oligonucleotide sequence
tatttcaacatattgctatggtccgacttttcagtaggca

SEQ ID NO: 17 - DNA, 25 bp, Artificial Sequence
  BL uni oligonucleotide sequence
atcaatcgcgtttacttgtcagcgg

SEQ ID NO: 18 - DNA, 50 bp, Artificial Sequence
  B100A oligonucleotide sequence
caatatgttgaaatagtccttaagtctggacaacttctcaccaaaatgtg

SEQ ID NO: 19 - DNA, 55 bp, Artificial Sequence
  B105 oligonucleotide sequence
caatatgttgaaatatcactgtccttaagtctggacaacttctcaccaaaatgtg

SEQ ID NO: 20 - DNA, 60 bp, Artificial Sequence
  B110 oligonucleotide sequence
caatatgttgaaatattgaatcactgtccttaagtctggacaacttctcaccaaaatgtg

SEQ ID NO: 21 - DNA, 40 bp, Artificial Sequence
  B190 oligonucleotide sequence
caatatgttgaaataccaggaagcactcacttctgaggtt

SEQ ID NO: 22 - DNA, 42 bp, Artificial Sequence
  B290 oligonucleotide sequence
tagctgccgggatccagactagaggttctcacttctgaggtt

SEQ ID NO: 23 - DNA, 47 bp, Artificial Sequence
  B295 oligonucleotide sequence
tagctgccgggatccagactagaggttaagcactcacttctgaggtt

SEQ ID NO: 24 - DNA, 46 bp, Artificial Sequence
  B300A oligonucleotide sequence
cttgaatcactgtccttaagtctggacaacttctcaccaaaatgtg

SEQ ID NO: 25 - DNA, 54 bp, Artificial Sequence
  B300B oligonucleotide sequence
aaggtttagaggttgcgcgcccatgcatgtcaggtttgggaccgactagaacgt

SEQ ID NO: 26 - DNA, 52 bp, Artificial Sequence
  B300C oligonucleotide sequence
tagctgccgggatccagactagaggttccaggaagcactcacttctgaggtt

SEQ ID NO: 27 - DNA, 52 bp, Artificial Sequence
  B300C oligonucleotide sequence
gcgggatatacgcctggcagcgaactagggtactcctctcaaccgtccgagg

SEQ ID NO: 28 - DNA, 46 bp, Artificial Sequence
  B300E oligonucleotide sequence
caatatgttgaaatatccacaactaatttatacagggatgttgatt

SEQ ID NO: 29 - DNA, 25 bp, Artificial Sequence
  BR uni oligonucleotide sequence
tgcctactgaaaagtcggaccatag

SEQ ID NO: 30 - DNA, 25 bp, Artificial Sequence
  primer sequence
nngctgacaagtaaacgcgattgnn

SEQ ID NO: 31 - DNA, 25 bp, Artificial Sequence
  primer sequence
ccgctgacaagtaaacgcgattgat

SEQ ID NO: 32 - DNA, 24 bp, Artificial Sequence
  primer sequence
cgctgacaagtaaacgcgattgat

SEQ ID NO: 33 - DNA, 22 bp, Artificial Sequence
  primer sequence
gctgacaagtaaacgcgattga

SEQ ID NO: 34 - DNA, 24 bp, Artificial Sequence
  primer sequence
ccgctgacaagtaaacgcgattga

SEQ ID NO: 35 - DNA, 23 bp, Artificial Sequence
  primer sequence
ccgctgacaagtaaacgcgattg

SEQ ID NO: 36 - DNA, 23 bp, Artificial Sequence
  primer sequence
cgctgacaagtaaacgcgattga

SEQ ID NO: 37 - DNA, 22 bp, Artificial Sequence
  primer sequence
cgctgacaagtaaacgcgattg

SEQ ID NO: 38 - DNA, 22 bp, Artificial Sequence
  primer sequence
gctgacaagtaaacgcgattga

SEQ ID NO: 39 - DNA, 22 bp, Artificial Sequence
  primer sequence
gctgacaagtaaacgcgattga

SEQ ID NO: 40 - DNA, 21 bp, Artificial Sequence
  primer sequence
gctgacaagtaaacgcgattg

SEQ ID NO: 41 - DNA, 25 bp, Artificial Sequence
  primer sequence
nngctgacaagtaaacgcgattgnn

SEQ ID NO: 42 - DNA, 24 bp, Artificial Sequence
  primer sequence
ngctgacaagtaaacgcgattgnn

SEQ ID NO: 43 - DNA, 23 bp, Artificial Sequence
  primer sequence
gctgacaagtaaacgcgattgnn

SEQ ID NO: 44 - DNA, 24 bp, Artificial Sequence
  primer sequence
nngctgacaagtaaacgcgattgn

SEQ ID NO: 45 - DNA, 23 bp, Artificial Sequence
  primer sequence
nngctgacaagtaaacgcgattg

SEQ ID NO: 46 - DNA, 25 bp, Artificial Sequence
  primer sequence
nncctactgaaaagtcggaccatnn

SEQ ID NO: 47 - DNA, 24 bp, Artificial Sequence
  primer sequence
gcctactgaaaagtcggaccatag

SEQ ID NO: 48 - DNA, 23 bp, Artificial Sequence
  primer sequence
cctactgaaaagtcggaccatag

SEQ ID NO: 49 - DNA, 24 bp, Artificial Sequence
  primer sequence
tgcctactgaaaagtcggaccata

SEQ ID NO: 50 - DNA, 23 bp, Artificial Sequence
  primer sequence
tgcctactgaaaagtcggaccat

SEQ ID NO: 51 - DNA, 23 bp, Artificial Sequence
  primer sequence
gcctactgaaaagtcggaccata

SEQ ID NO: 52 - DNA, 22 bp, Artificial Sequence
  primer sequence
gcctactgaaaagtcggaccat

SEQ ID NO: 53 - DNA, 22 bp, Artificial Sequence
  primer sequence
gcctactgaaaagtcggaccat

SEQ ID NO: 54 - DNA, 22 bp, Artificial Sequence
  primer sequence
cctactgaaaagtcggaccata

SEQ ID NO: 55 - DNA, 21 bp, Artificial Sequence
  primer sequence
cctactgaaaagtcggaccat

SEQ ID NO: 56 - DNA, 25 bp, Artificial Sequence
  primer sequence
nncctactgaaaagtcggaccatnn

SEQ ID NO: 57 - DNA, 24 bp, Artificial Sequence
  primer sequence
ncctactgaaaagtcggaccatnn

SEQ ID NO: 58 - DNA, 23 bp, Artificial Sequence
  primer sequence
cctactgaaaagtcggaccatnn

SEQ ID NO: 59 - DNA, 24 bp, Artificial Sequence
  primer sequence
nncctactgaaaagtcggaccatn

SEQ ID NO: 60 - DNA, 23 bp, Artificial Sequence
  primer sequence
nncctactgaaaagtcggaccat